(19) United States
(12) Patent Application Publication
(10) Pub. No.: US 2010/0048738 A1
(43) Pub. Date: Feb. 25, 2010

(54) HYBRID COMPOUNDS BASED ON POLYOL(S) AND AT LEAST ONE OTHER MOLECULAR ENTITY, POLYMERIC OR NON-POLYMERIC, IN PARTICULAR OF THE POLYORGANOSILOXANE TYPE, PROCESS FOR THE PREPARATION THEREOF, AND APPLICATIONS THEREOF

(75) Inventors: Etienne Fleury, Irigny (FR); Sami Halila, Voreppe (FR); Hugues Driguez, Grenoble (FR); Sylvain Cottaz, Herbeys (FR); Thierry Hamaide, Vienne (FR); Sebastien Fort, Uriage (FR)

Correspondence Address: PEARNE & GORDON LLP, 1801 EAST 9TH STREET, SUITE 1200, CLEVELAND, OH, 44114-3108, US

(73) Assignees: RHODIA OPERATIONS, Aubervilliers, FR (FR); CENTRE NATIONAL DE LA RECHERCHE SCIENTIFIQUE-CNRS, Paris (FR)

(21) Appl. No.: 12/300,470

(22) PCT Filed: May 15, 2007
(86) PCT No.: PCT/EP2007/054702

§ 371 Date: Nov. 6, 2009

(30) Foreign Application Priority Data
May 15, 2006 (EP) 06 51744
May 15, 2006 (EP) 06 51745

(51) Int. Cl. A61K 8/06; C08G 77/38; C07H 5/04; C11D 1/00

(52) U.S. Cl. 514/772.7; 525/54.2; 536/29.11; 510/417; 510/122; 510/119

(57) ABSTRACT

The invention relates to novel hybrid compounds comprising at least one polyon entity (Po)—for example oligomer or polymer—in which at least one of the hydroxyl functions of Po is substituted by at least one entity A that can be of a variable nature, for example polymer (e.g. polyorganosiloxane-POS), hydrocarbonated or mineral. The bond Ro between the entity Po and the entity A is obtained by means of “click chemistry” and corresponds to formula (II.1) or (II.2), Z representing —CH— or —N—. A is an entity selected from the group comprising the various polyols of Po, polyorganosiloxanes (POS), polyalkylene glycols, polyamides, polyesters, polystyrenes, alkyls, alkenyls, alkynyls, aryls, and combinations thereof, in addition to mineral materials such as silica and the combinations thereof. Said hybrid components can be used as emulsifiers, especially for cosmetics.

### FIELD OF THE INVENTION

The present invention relates to novel hybrid structures containing at least one polyol entity (Po)—for example oligomeric or polymeric—and at least one entity A which can be of varied nature, for example polymeric (e.g. polyorganosiloxane-POS), hydrocarbon or inorganic. The bond or bonds between said entity Po and said entity A are obtained according to the chemical mechanism designated by the name “click chemistry”, in which an azide (or nitride) reactive unit reacts with a reactive unit of the alkynyl or nitrile type, to form a linking hinge (Ro) of the triazole or tetrazole type.

The invention also relates to the process for obtaining of these hybrid structures as well as their applications as amphiphilic compounds, for example.

Finally, the invention also relates to the synthons, i.e. intermediate products bearing functional groups of the azide and/or alkynyl and/or nitrile type and involved in the preparation of these hybrid structures.

### TECHNOLOGICAL BACKGROUND AND PRIOR ART

The polyols more particularly, but not limitatively, covered by the present invention (entity Po), contain oligosaccharides or polysaccharides (linear, branched or cyclic) at least in part made up of at least two, preferably at least three monosaccharide units, linked together by oside linkages.

These specific polyol polymers Po which are polysaccharides are of some interest on account of their physicochemical properties (hydrophilic, hydrolysable, bioresorbable etc.), their chemical complexity offering multiple possibilities in terms of structure and properties, their high availability and their natural origin, inter alia. This natural origin can render them particularly attractive from an environmental and/or toxicological and/or commercial point of view. Thus, the uses of polysaccharides such as starch products and derivatives thereof or cellulose products and derivatives thereof are very numerous.

The POS (silicones) constitute another class of polymers of major interest in many branches of industry. In addition to the fact that the POS are readily obtainable raw materials, they are also characterized by their hydrophobic properties.

This led to the idea of creating hybrid structures based on polysaccharides and POS, so as to have available, for example, emulsifying compounds that can be used in particular in cosmetic compositions such as for example compositions for skin care, compositions for sun protection and treatment, shampoo compositions and deodorant and/or antiperspirant compositions, for example in “stick”, gel or lotion form, inter alia.

In these particular hybrid structures which are the polysaccharide-POS systems, the polysaccharide entity and the POS entity combine their respective advantages.

The polysaccharide entity, owing to the presence of its many hydroxyl groups, can enter into strong intra- or intermolecular interactions, both in a hydrophobic medium and in a hydrophilic medium. This molecular recognition type of behaviour makes it possible to obtain structures of the gel type and/or to promote interactions with polar surfaces such as textiles (i.e. cotton) or the hair.

The POS entity contributes two major advantages. The first is the flexibility which endows this POS entity with high reactivity and an ability to adapt its molecular conformation depending on the substrate or substrates present. The second advantage, among others, is due to the hydrophobic nature of this POS entity which contributes low surface energy properties.

As an example of a commercial product containing hybrid polysaccharide-POS structures, that distributed under the name “Wacker-Belsil® SPG 128 VP” may be cited. This is a cyclopentadimethylsiloxane one part of the siloxy D units whereof is substituted with a polyglucoside chain linked to the silicon by a linking hinge containing two oxygen bridges and another part of the D units whereof is substituted with an alkyl radical of the type —(CH₂)_{w}—CH₃, w being a natural whole number.

Throughout the present document, reference will be made to conventional nomenclature for denoting the M, D, T and Q groups of the POS. As a reference work NOLL “Chemistry and technology of silicones”, Chapter 1.1, pages 1-9, Academic Press, 1968—2nd edition can be mentioned.

It is known that the preparation of hybrid polysaccharide-POS systems can be carried out by grafting of polysaccharide entities onto a POS entity according to two main approaches: hydrosilylation or condensation.

By way of illustration of the hydrosilylation route, two prior patent references, namely EP-B-O 612 759 and WO-A-2005/087843, can for example be cited.

EP-B-O 612 759 describes organosilicon compounds containing a glycoside residue obtained by reacting an alkenylated mono or polysaccharide (1 to 10 monosaccharide units) with a POS, for example a disiloxane, bearing SiH groups. The alkenyl group is introduced directly onto the oligosaccharide or polysaccharide, unprotected, in the anomeric position, by means of alkyl oxyethanol, in the presence of a strong acid, at 100° C. The hydrosilylation is carried out by means of a Speier platinum catalyst in isopranol at 100° C. The hybrid compound obtained in the examples corresponds to the following formula:

H(C₆H₁₀O₅)_{1.5}—O—CH₂CH₂O—CH₂—CH₂—CH₂—Si(CH₃)₂—O—Si(CH₃)₃

WO-A-2005/087843 describes a graft polymer containing a polyorganosiloxane skeleton and glycoside units (mono and/or polysaccharide). In particular, WO-A-2005/087843 describes the preparation of a polydimethylsiloxane grafted with a cellobiose functionalised with an allyl unit. In order to do this, the cellobiose is reacted with allylamine. After attachment of the allylamine unit to the anomeric carbon of the cellobiose, the amine group and some of the primary hydroxyls are protected by acetylation. As for the remaining hydroxyl groups, these are protected by substitution of their hydrogen with a trimethylsilyl unit. The hydrosilylation of the polydimethylsiloxane with dimethylhydrogenosiloxy ends is then carried out in presence of Karstedt platinum at a temperature of 70° C. Deprotection of the POS grafted at its ends with the disaccharide cellobiose is then carried out by means of a tetrahydrofuran/methanol mixture in an acidic medium. The reaction scheme is as follows:

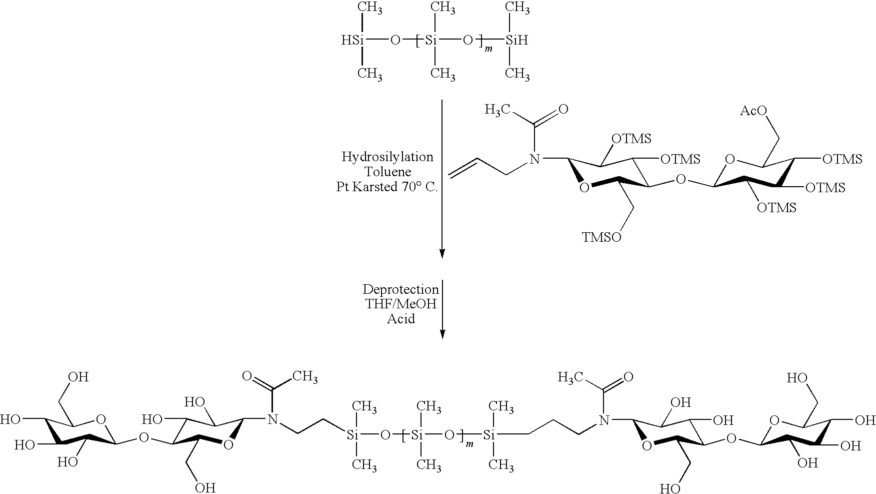

The protection/deprotection constraints of the saccharides are an appreciable drawback of these known polyorganosiloxane/glycoside graft polymers and the process for obtaining them.

Apart from grafting by hydrosilylation and condensation, the U.S. Pat. No. 5,428,142 which describes the grafting at C1 of an unprotected sugar, onto the terminal primary alcohol group, of polyoxyethylene grafts attached to a polysiloxane chain, by etherification in a strongly acidic medium at 100° C. can be cited anecdotally.

Also known is the mechanism for chemical linkage called “click chemistry” or the Huisgen reaction. Huisgen and Szeimies [(a) Huisgen, R.; Szeimies, G.; Moebius, L. Chem. Ber. 1967, 100, 2494. (b) Huisgen, R.; Knorr, R.; Moebius, L.; Szeimies, G. Chem. Ber. 1965, 98, 4014] were the first to carry out the 1,3-dipolar cycloaddition of an azido derivative to an alkyne derivative at high temperature. The reaction scheme for this cycloaddition is as follows:

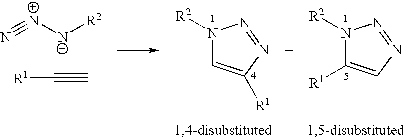

The patent application WO-A-03/101972 describes the cycloaddition reaction (so-called “Huisgen” reaction), between azides and alkynes, in presence of a copper I catalyst. This reaction makes it possible to form, regiospecifically, essentially the 1,4-disubstituted 1,2,3-triazole. As shown in FIGS. 3A and 3B of WO-A-03/101972, this 1,3-dipolar cycloaddition makes it possible to obtain, for example, hybrid systems (cf. products 1 to 10) containing on the one hand phenyl nuclei and on the other hand inert or branched cyclic molecules, optionally unsaturated and optionally bearing hydroxyls, as well as a hybrid system (11) containing a triazole hinge linking on the one hand a propanediol residue and, on the other hand, a polycyclic dihydroxy compound. Furthermore, it follows from FIGS. 6 to 8 of WO-A-03/101972 that it is possible to functionalise biological amine molecules such as erythromycin (cf. FIG. 6), and also molecules containing polyazide or polyalkyl nuclei (cf. FIGS. 7 and 8).

WO-A-03/101972 does not mention hybrid compounds containing polyol entities linked by “click chemistry” cycloaddition to different polyol entities or to POS, polyalkylene glycol, polyamide, polyester, alkyl, alkenyl, alkynyl or aryl entities and combinations thereof, nor to inorganic substances such as silica.

The application WO-A-2005/118625 describes other applications of 1,3-dipolar cycloaddition “click chemistry” aiming to produce hybrid systems containing an entity A corresponding to a carbohydrate linked by a 5-membered cyclic 1,2,3-triazole hinge to an entity B consisting of an amino acid or an amino acid analogue or to an entity C representing a polypeptide or a polypeptide analogue. These hybrid systems are obtained by reacting the carbohydrate functionalised with an acetylene group or with an azide and an amino acid or a polypeptide functionalised with a corresponding amide or acetylene group. In the sense of WO-A-2005/118625, the term carbohydrate (cf. p. 7, 1.20 to p. 8, 1.2) designates both mono- and also polysaccharides, in which the hydroxy groups may be replaced by hydrogen, by an amine or thiol group, or by groups of heteroatoms. The 1,3-dipolar cycloaddition is carried out by protecting the hydroxy groups of the saccharide with an acetyl group, and the amine group of the amino acid with a Boc group, and by using a copper-containing catalyst and diisopropyl-ethylamine, in a tetrahydrofuran solvent medium. The pseudo-glycoamino acids and glycopeptides obtained can be used for the treatment of bacterial diseases.

It should be noted that in the systems AB or AC according to WO-A-2005/118625, the substitution of the “carbohydrate” entity A with B or C takes place exclusively on the anomeric carbon of A. Furthermore, the 5-membered cyclic 1,2,3-triazole hinge is linked directly by a covalent bond to this anomeric carbon, with no spacer unit. Finally, the constraint of protection of the sensitive groups (OH, amine) of A, B and C which is necessary in the synthesis of the systems AB or AC according to WO-A-2005/118625 is extremely disadvantageous, in particular at the industrial level.

It must therefore be concluded that the preparation of hybrid systems by “c lick chemistry” or 1,3-dipolar cycloaddition of an azide derivative to an alkyne derivative in the presence of copper is limited to the combination of (poly)-saccharide polyols with amino acids or (poly)peptides.

One of the essential objectives of the present invention is to provide other hybrid compounds obtained by “click chemistry”.

Another essential objective of the invention is to provide novel hybrid compounds based on polyol entity (entities) Po linked by at least one triazole or tetrazole pentacyclic hinge to at least one entity A, these hybrid compounds being capable of being used in many applications, both industrial (emulsifiers) and biological.

Another essential objective of the invention is to provide hybrid compounds containing one or more (polymer)-polyols, for example polysaccharide entities Po, linked by pentacyclic triazole or tetrazole hinges obtained by “click chemistry” to at least one entity A of (polymer)-polyols type and/or POS and/or polyalkylene glycol, and/or polyamine (peptides), and/or polyester, and/or polystyrene, and/or alkyl, and/or alkenyl, and/or alkynyl, and/or aryl, and/or inorganic such as silica.

Another essential objective of the invention is to provide hybrid polysaccharide/POS compounds linked by at least one hinge derived from a 1,3-dipolar cycloaddition of an azide or nitrile derivative to an alkyne derivative, with copper catalysis by “click chemistry”.

Another essential objective of the invention is to provide hybrid compounds containing one or more (polymer)-polyol entities Po, for example polysaccharide, these compounds being capable of being prepared without laborious stages of protection/deprotection of the reactants, in particular of the saccharides.

Another essential objective of the invention is to provide a simple process of preparation of hybrid compounds containing one or more (polymer)-polyols, for example polysaccharide, entities Po, in particular without laborious stages of protection/deprotection of the reagent, in particular of the saccharides.

Another essential objective of the invention is to provide cosmetic compositions, shampoo compositions and cleaning compositions, containing hybrid compounds as defined in the above objectives.

These aims, among others, are attained by the present invention, which relates first of all to a hybrid compound Po-Ro-A containing at least one polyol entity (Po) and in which at least one of the atoms of Po is substituted with at least one group of the following general formula (I):

-Ro-A (I)

in which:
Ro is a linking hinge of the following formula (II.1) or (II.2):

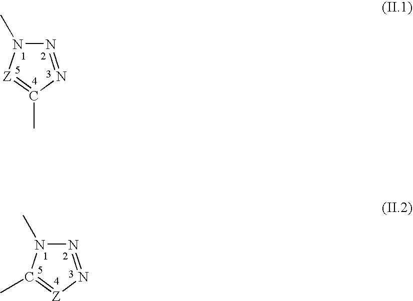

with Z representing either a carbon atom for example substituted by a hydrogen (not shown in formulae (II.1) and (II.2) by convention) or a nitrogen atom;
the polyol entity Po is selected from the polymeric synthetic non-saccharide polyols, or from the saccharides (hydrogenated or not) containing at least two, preferably at least three monosaccharide units,
A is an inorganic or organic entity, optionally polymeric; and if there is a plurality of entities A per molecule of hybrid compound, said entities A are mutually identical to or different, the organic entity A being selected or derived from a compound selected from the group comprising:
   synthetic polymers, their copolymers or monomer units making it possible to obtain them
   alkyls, alkenyls, alkynyls, aryls and combinations of the latter
   and combinations thereof.

Within the meaning of the invention, the term “hybrid” designates homogeneous (Po is identical to A) or heterogeneous (Po is different from A) Po-Ro-A structures.

It should be noted that if Z represents a carbon atom, it is also linked to a hydrogen atom (not usually shown), so as to satisfy the valence of said carbon.

Preferably, at least one of the hydroxyl functions of the polyol entity (Po) is substituted with at least one grouping with the following general formula (I): -Ro-A as defined above.

According to one embodiment M1 of the invention, the hybrid compound according to the invention is characterized in that the hinge Ro or at least one of the hinges Ro is linked to the entity Po and/or to the entity A by a divalent linkage -L-. In other words, L is a spacer unit.

Advantageously, L can for example be a hydrocarbon unit or an atom such as O or S. Within the meaning of the invention, the term “hydro(geno)carbon unit” designates a unit containing for example at least one carbon atom and/or at least one hydrogen. This includes in particular the “ester”, “amide”, “imine”, bonds . . . .

According to one embodiment M2 of the invention, the hybrid compound according to the invention is characterized in that none of the hinges Ro is linked to the entity Po by a divalent linkage -L- and in that said hybrid compound contains at least one entity A free from amino acid(s) and/or peptide(s) and/or their analogue(s) and/or derivative(s).

These novel hybrid compounds are easy to construct at an acceptable cost. They are therefore perfectly suitable for industrial use and they open the way to a large number of uses, in particular in the amphiphilic ingredients sector, being utilisable in particular in cosmetics or as detergents, for example: cosmetic care compositions, creams, lotions, gels, deodorant and antiperspirant compositions, soap compositions, shampoo compositions, washing compositions, etc.

The particular hybrid compounds which are polysaccharide-Ro—POS and polysaccharide-Ro-alkyl combinations represent a novel group of structures which are particularly interesting in terms of compatibility with industrial requirements, in particular relating to cost and environmental impact and in terms of use.

The present invention also proposes a novel process for obtaining the aforementioned hybrid compounds. This process is characterized in that:

i. a synthon Po-X, containing at least one reactive unit X having at least one reactive end of formula (VII.1): —C≡E; with E=CH or N is used and/or is prepared;
ii. a synthon A-Y containing at least one reactive unit Y having at least one reactive end of formula (VII.2); —N₃; the reactive end (VII.2) being capable of reacting with the reactive end (VII.1) is used and/or is prepared;
iii. the synthon Po-X is reacted with the synthon A-Y according to a cycloaddition mechanism, so as to obtain a hybrid compound Po-Ro-A containing at least one polyol entity (Po) in which at least one of the hydroxyl functions of Po is substituted with at least one grouping of the following general formula (I′): -Ro-A; with Ro and A as defined above;
iv. optionally, Po-Ro-A is separated from the reaction mixture in such a manner as to recover it.

Such a process is particularly advantageous because of its simplicity, its economy, its ecocompatibiity and the multiplicity (variety) of products that it makes it possible to obtain.

It should be noted that, according to a variant, instead of or as well as the synthons Po-X and the synthons A-Y, it is possible to use mixed synthons Po-XY each containing at least one reactive unit X and at least reactive unit Y and mixed synthons A-XY each containing at least one reactive unit X and at least one reactive unit Y, such that said synthons Po-XY and A-XY are capable of reacting together or indeed with themselves.

The invention also relates to:
synthons Po-X containing at least one reactive unit X having at least one reactive end of formula (VII.1.1):

-[L₁]ₐ-C≡E
with E=CH or N, a=0 or 1, the said end being linked to the residue Po by a linkage L₁ which is a divalent hydrocarbon linkage;
synthons Po-Y containing a reactive unit Y having at least one reactive end of formula (VII.2.1):

-[L₂]ₐ-N₃
with a=0 or 1; said end being linked to the residue Po by a linkage L₂ which is a divalent hydrocarbon linkage;
synthons A-X containing a reactive unit X having at least one reactive unit X having at least one reactive end of formula (VII.1.3):

-[L₃]ₐ-C≡E
with E=CH or N, a=0 or 1 (if a=0, then A is different from a saccharide or a peptide and if a=1, then A is different from a PDMS), said end being linked to the residue A by a linkage L₃ which is a divalent hydrocarbon linkage;
synthons A-Y containing a reactive unit Y having at least one reactive end of formula (VII.2.4):

-[L₄]ₐ-N₃
with a=0 or 1 (if a=0, A is different from a saccharide or a peptide), said end being linked to the residue A by a linkage L₄ which is a divalent hydrocarbon linkage;
mixed synthons Po-XY in which the reactive units X and Y correspond to the same definitions as those given above for the synthons Po-X and Po-Y;
or mixed synthons A-XY in which the reactive units X and Y correspond to the same definitions as those given above for the synthons A-X and A-Y.

In the above formulae (VII.1.1), (VII.2.1), (VII.1.3), (VII.2.4) of the synthons Po-X, Po-Y A-Y, A-X, if a=0, then there is no linkage L₁, L₂, L₃, L₄ (or spacer unit), but a direct valence bond (e.g. covalent bond).

These synthons are useful, novel and effective intermediate products for the implementation of the aforementioned process and for obtaining the hybrid compounds according to the invention.

Finally, the invention relates to the uses of these hybrid compounds and the compositions containing them.

### DETAILED DESCRIPTION OF THE INVENTION

### The Compound Po-Ro-A

The linking hinge Ro of formula (II.1) or (II.2) is at the heart of the hybrid compounds according to the invention.

This linking hinge is the result of a “click chemistry” reaction, i.e. 1,3-dipolar cycloaddition, on the one hand, of an azido derivative, the reactive end of which bears three nitrogen atoms, and on the other hand, of an alkyne derivative (Z=C) or of a nitrile derivative (Z=N).

This linking hinge Ro is a 5-membered, 1,4-disubstituted (cf. formula II.1) or 1,5-disubstituted (cf. formula II.2) triazole (Z=C) or tetrazole (Z=N) heterocycle.

Depending on whether the reactive functional groups of the azido type, on the one hand, and of acetylenic or nitrile type on the other hand, are borne by the entity Po or the entity A, this gives rise to hybrid compounds of different structures.

Thus, according to a first structure, the free valence bond of the nitrogen at the 1 position in formulae (II.1) and (II.2) links the hinge Ro to Po and the free valence bond of the carbon or of the atom Z in 4 or 5 position in formulae (II.1) and (II.2) links the hinge Ro to A.

According to a second structure, the free valence bond of the nitrogen at the 1 position in formulae (II.1) and (II.2) links the hinge Ro to A and the free valence bond of the carbon or of the atom Z at the 4 or 5 position in formulae (II.1) and (II.2) links the hinge Ro to Po. Naturally, the hybrid compounds according to the invention are not limited to compounds containing just a single linking hinge Ro but also cover hybrid compounds each containing several mutually identical or different linking hinges Ro.

These structures with several mutually identical or different linking hinges Ro, refer for example to branched multibridge products, e.g. of the dendrimer type, in star or other shapes . . . .

In particular, in the embodiment M1 according to which the hinge Ro or at least one of the hinges Ro is linked to the entity Po by a divalent linkage -L-, the latter can in particular contain at least one of the linkages L₁, L₂, L₃, L₄, as defined above in formulae (VII.1.1), (VII.2.1), (VII.1.3), (VII.2.4) of the synthons Po-X, Po-Y A-Y, A-X. In other words, L is a spacer unit. Inter alia, the simplified general formulae of the corresponding hybrid compounds can be those belonging to the group comprising: Po-L₁-Ro-L₂-Po; Po-L₁-Ro-L₄-Po; Po-L₂-Ro-L₃-A; A-L₃-Ro-L₄-A; L₁, L₂, L₃, L₄ are spacer units and are mutually identical to or different, whether they are taken separately or together.

A is an inorganic or organic entity, optionally polymeric; and in the case of a plurality of entities A per molecule of hybrid compound, the said entities A are mutually identical to or different, the organic entity A being selected or derived from a compound selected from the group comprising:
synthetic polymers, their copolymers or monomer units making it possible to obtain them
alkyls, alkenyls, alkynyls, aryls and combinations of the latter
and combinations thereof.

The synthetic polymers of the entity A can be synthetic polymers of average molar mass greater than 1000 g/mol, preferably greater than 10000 g/mol.

In practice, A is selected or derived from a compound selected from the group comprising:
synthetic polymeric non-saccharide polyols, their copolymers or the monomer units making it possible to obtain them (details of some are given later for the entity Po);
polyorganosiloxanes (POS), their copolymers or the monomer units making it possible to obtain them;
polyalkylene glycols (or else polyoxides of alkylenes), preferably polyethylene glycols (or else ethylene polyoxides) and/or polypropylene glycols (or else propylene polyoxides), and/or polytetraethylene glycols, and/or their copolymers or co-oligomers, in particular random or block copolymers or co-oligomers or of polypropylene glycols and of polypropylene glycols (or else random or block polyoxides of ethylene and of propylene), these polyalkylene glycols being optionally functionalized with or onto other groups, for example with or onto amine groups (Jeffamines), and/or being optionally terminated at least one end by a hydroxyl group or by an alkyl group, for example a C₁-C₃₀ alkyl;
polyamides, their copolymers or the monomer units making it possible to obtain them;
polyesters, their copolymers or the monomer units making it possible to obtain them;
polybutadienes, their copolymers or the monomer units making it possible to obtain them;
polystyrenes, their copolymers or the monomer units making it possible to obtain them;
optionally, alkyls, alkenyls, alkynyls, aryls and combinations of these;
inorganic substances such as silica;
and combinations thereof.

It may be advantageous for this entity A to contain polymers or copolymers selected from the group as mentioned above, or else linear or branched chains, optionally cross-linked. For example, the molar mass of this entity A is greater than or equal to 100, preferably greater than or equal to 100, and still more preferably between 100 and 50000.

According to a preferred embodiment of the invention, the entity A contains at least one POS bearing siloxy units M, D, T and/or Q, preferably at least one POS bearing siloxy units M and D, optionally T and/or Q, and still more preferably at least one POS of type M(D)_{d}M, M(D)_{d}(T)ₜM, MQ, with d and t being rational numbers greater than or equal to 0. d is for example between 1 and 1,000,000, preferably from 1 to 10,000 and t is for example between 0 and 50, preferably between 0 and 20.

In practice, these POS are for example α, ω functional, linear polysiloxanes or they are functionalized in the chain. These POS can also be structures with a varied degree of branching. In practice, these POS bear, for example, glycidyl ether function(s) and/or hydrogen.

According to a particular sub-embodiment of the invention, the hybrid compound Po-Ro-POS corresponds to at least one of the following formulae:

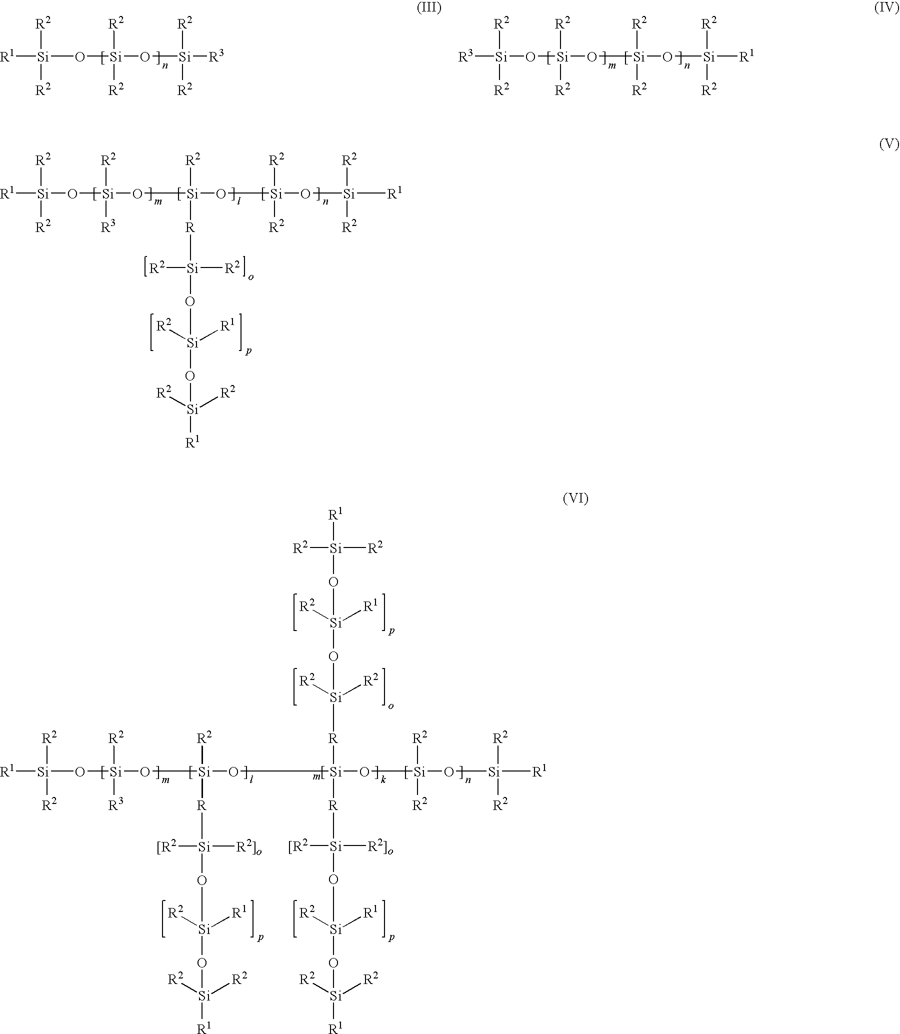

in which:
R², identical or different, is a hydrocarbon group, preferably a methyl group,
R³, identical or different, is a group of formula -Ro-Po in which Ro and Po are as defined above,
R¹, identical or different, is a group R² or R³,
R is a divalent group comprising an oxygen atom, preferably an —O— group,
m is an average number different from 0,
n is an average number greater than or equal to 0,
k and l are average numbers greater than or equal to 0, and
o and p, identical or different, are average numbers greater than or equal to 0.

Preferably,
m+n lies between 0 and 1000000, preferably between 0 and 10000, the ratio of m to n lying between 1/1 and 1/100, preferably between 1/20 and 1/50, or
m+n+o+p lies between 0 and 1000, preferably between 0 and 300, the ratio of n+o to m+p lying between 1/1 and 1/100, preferably between 1/20 and 1/50.

The entity A can also include residues of the polyalkylene glycol type optionally having at least one alkyl ether terminus, for example methyl ether.

As examples of polyalkylene glycols, polyoxyethylene glycols, monoalkyl (e.g. methyl)ether polyoxyethylene glycols, polyoxypropylene glycols, monoalkyl (e.g. methyl)ether polyoxypropylene glycols, polyoxytetraethylene glycols etc. can be mentioned.

Polyamides can be constituent elements of entity A. As examples of polyamides, polyamides 6-6, polyamides 6, polyamides 6 monoamine, polyamines 6-10, polyamides 12-12 etc can be mentioned.

Polyesters can be constituent elements of entity A. As examples of polyesters, poly ε-caprolactone, polylactic acid, ethylene glycol polyadipate, polyhydroxyalkanoate etc can be mentioned.

Polystyrenes can be constituent elements of entity A. As examples of polystyrenes, hydroxytelechelic or monofunctional polystyrene etc can be mentioned.

Polybutadienes can be constituent elements of entity A. As examples of polybutadienes, hydroxytelechelic polybutadiene etc can be mentioned.

Amino acids and peptides can be constituent elements of the entity A. Within the meaning of the invention, the term “peptides” designates, inter alia, oligopeptides and polypeptides, or even proteins. Derivatives (or analogues) of amino acids (natural or synthetic) and of peptides are also targeted by the invention as the entity A.

All the (co)polymers capable of entering into the constitution of entity A of the hybrid compound Po-Ro-A can be linear or branched or cross-linked homopolymers, or else, linear or branched, optionally cross-linked, random or block copolymers.

When A is a (co)polymer, it can be envisaged that the synthon A-X or A-Y used to prepare the hybrid compound, may contain a finished (co)polymer or an unfinished monomer, oligomer or polymer unit, destined to grow to form a finished polymer after reaction with Po-Y or Po-X.

The alkyl, alkenyl or alkynyl chains capable of being included in entity A for example contain from 2 to 50 carbon atoms, preferably from 4 to 40, and more preferably from 4 to 30 carbon atoms. As examples, butyl, octyl, dodecyl, octadecyl, eicosan etc. can be mentioned.

Silica is an example of an inorganic material capable of entering into the constitution of the entity A.

Regarding the polyol entity Po, it is selected from the synthetic polymeric, non-saccharide polyols, and/or from the saccharides (hydrogenated or not) containing at least two, preferably at least three monosaccharide units.

The synthetic polymeric, non-saccharide polyols can in particular have an average molar mass greater than 1000 g/mol, preferably greater than 10000 g/mol. The latter are, for example, polyvinyl alcohols (partially hydrolysed or not), polyhydroxyaldehydes H-[CHOH]ₙ—CHO and/or polyhydroxyketones H—[CHOH]ₙ—CO—[CHOH]ₘ—H preferably containing at least 3, more preferably at least 4 carbon atoms. The synthetic polymeric, non-saccharide polyols preferably have at least 3, more preferably at least 4, and still more preferably at least 10 hydroxyl units. They preferably have at least 3, more preferably at least 4, and still more preferably at least 10. Note that they can constitute entities A repeating units.

Concerning the “saccharides” (also called “carbohydrates”), it must be clearly stated that, in the context of the invention, the generic term “saccharide” includes, it will have been understood, monosaccharides, disaccharides, oligosaccharides and polysaccharides as well as all the derivatives of the saccharides.

The saccharides, their structures and formulae are known to the person skilled in the art. In particular, it is known that the saccharides have a non-reducing end and a reducing end. The latter involves the presence of an “anomeric hydroxyl”, and is situated on the right according to the writing convention. It is also known that the saccharides have —OH groups. According to the invention, when Po contains a saccharide, the carbon of the saccharide more preferably contained in the bond(s) with the hinge(s) Ro is the “anomeric” carbon. This does not exclude the fact that all or some of the other saccharide carbons can be linked to a hinge Ro. This is all the more possible when the groups borne by the “non-anomeric” carbons do not require protection during the synthesis of the hybrid compound. The monosaccharides are molecules containing a single saccharide unit (for example C5: pentose or C6: hexose), with no glycosidic connection between several units of this type. The monosaccharides include inter alia the aldoses, dialdoses, aldoketoses, ketoses and diketoses, as well as the deoxysaccharides, aminosaccharides and derivatives thereof resulting from precursors at least potentially containing a carbonyl group.

As examples of monosaccharides, the following saccharides are mentioned:

D-glucose, fructose, sorbose, mannose, galactose, talose, allose, gulose, idose, glucosamine, mannosamine, galactosamine, glucuronic acid, rhamnose, arabinose, galacturonic acid, fucose, xylose, lyxose, ribose.

As examples of di- or oligo-saccharides, the following saccharides are mentioned:
di-saccharides: maltose, gentiobiose, lactose, cellobiose, isomaltose, melibiose, laminaribiose, chitobiose, xylobiose, mannobiose, sophorose, palatinose
oligo-saccharides: maltotriose, isomaltotriose, maltotetraose, maltopentaose, xyloglucan, maltoheptaose, mannotriose, manninotriose, chitotriose, generally the di- or oligo-saccharides having, for example, β-1-4, α-1-4 or α-1-6 linkages, etc.

The polysaccharides according to the invention can be linear or branched and can contain for example more than 20 monosaccharide residues or preferably more than 30 monosaccharide residues or even more in particular between 25 and 100 monosaccharide residues. The latter may be mutually identical to or different.

The polysaccharides according to the invention can contain linear mono-, di-, tri-, tetra-, penta-, hexa-, hepta-, octa-, nona- or decasaccharide, preferably mono-, di-, tri- or tetra-saccharide, units. The polysaccharides can contain at least two, or at least three or at least four, or at least ten, or markedly more in the case of polysaccharide polymers, of these linear units.

In certain variants, the polysaccharides according to the invention can contain recurring saccharide units of the N-acetyl-lactosamine type or acetylated saccharide units.

Also mentioned as examples of polysaccharides are:
starch (preferably having at least 5 dextrose equivalents DE) and derivatives thereof such as the maltodextrins, cyclodextrins and glucose syrups,
pectin;
cellulose and derivatives thereof;
galactomannans, for example guar or carob polymers and derivatives thereof, [the macromolecule of guar or carob consists of a main linear chain made up of monomeric β-D-mannose sugars linked together by (1-4) bonds, and α-D-galactose side units linked to the β-D-mannoses by (1-6) bonds. Natural guar is extracted from the albumen of the seeds of certain plants for example Cyamopsis Tetragonalobus];
chitin and chitosan
bacterial polysaccharides
hyaluronic acid.

According to a particular embodiment the entity Po is different from a maltodextrin.
According to another particular embodiment the entity Po is different from a cyclodextrin.
According to another particular embodiment the entity Po is different from a glucose syrup.
According to yet another particular embodiment the entity Po is different from a maltodextrin and from a cyclodextrin and/or from a glucose syrup.

The starchy or cellulosic polysaccharides capable of entering into the constitution of the polyol entity Po are preferably of natural origin, but could also be obtained by a synthetic route.

As saccharide derivatives, the following can in particular be mentioned:
those obtained by reduction of the carbonyl group (alditol),
those obtained by oxidation of one or more terminal or non-terminal groups so as to transform them for example into carboxylic acid groups or into carboxyalkyl groups (e.g. carboxymethyl),
those obtained by grafting of one or more groups for example carboxylic acid groups, carboxyalkyl groups (e.g. carboxymethyl), hydroxyalkyl groups (e.g. hydroxyethyl) or indeed also alkyl groups (e.g. methyl);
those obtained by replacement of one or more hydroxy groups by a hydrogen atom, an amine group, a thiol group or a similar heteroatomic group;
those obtained by hydrogenation;
glycosides, namely compounds containing at least one saccharide and at least one aglycon (non-saccharide compound), saccharide(s) on the one hand, and non-saccharide component(s) on the other hand, being linked together by hydrolysable bonds
derivatives of galactomannans, in particular the derivatives of guar polymers or of carob polymers, obtained by hydrolysis of natural guar or carob, and optionally by chemical modification (derivatisation).

Derivatization can be used for chemically modifying derivatives of saccharides other than those mentioned above.

One of the major advantages of the invention is that of proposing hybrid compounds whose synthesis does not necessitate protection of sensitive groups, in particular those borne by the saccharides of the entity Po or A.

Naturally, such protection is nonetheless possible, for example to improve the solubility.

### The Process

According to another of its aspects, the invention relates to a process for obtaining hybrid compounds and in particular those according to the invention, such as described above.

This preparation process is that defined above. It comprises the four stages (i), (ii), (iii) and optionally (iv), which are described in detail below for non-limiting illustration.

### Stages (i) and (ii): the Synthons Used

More precisely in the case of the starting synthons Po-X:

1. according to a 1st possibility, Po has at least one saccharide with:
L₁ containing at least one amine group (for example terminal) having reacted with the anomeric carbon of the Po,
and/or L₁ derived from a precursor containing at least one halogeno group (for example bromo) having reacted with the OH group or groups of the Po;
   2. according to a 2nd possibility, Po contains at least one residue (for example saccharide) functionalized with at least one functionalising group belonging to the group comprising the carboxylic, carboxylate, anhydride, thiol, isocyanate and epoxide functionalising groups with:
L₁ containing at least one amine group (for example terminal) having reacted with the functionalising group(s) of the Po,
and/or L₁ derived from a precursor having at least one halogeno group (for example bromo) having reacted with the functionalising group(s) of the Po;
   3. according to a 3rd possibility, the first two possibilities are combined.

Advantageously, this synthon Po-X can be characterized in that Po is a polymer comprising, for example, at least two, preferably at least 3, and still more preferably at least 10 monomer units.

The preparation of the synthon Po-X can advantageously include the following essential substages:
a—reaction of the hydroxyl borne by the anomeric carbon of Po if the latter is saccharide and/or from the functionalising group or groups of Po with an excess of at least one precursor of the linkage L₁ bearing a reactive end (preferably, at least one amine function—optionally terminal—
b—elimination of the precursor;

According to a preferred characteristic, L₁ corresponds to —NH—(CH₂)_{q}≧1, with a precursor corresponding to:

NH₂CH₂_{q}C≡E

and still more preferably to propargylamine:

NH₂CH₂_{q}C≡CH

More precisely in the case of the starting synthons A-X:

1. according to a 1st possibility, A comprises at least one saccharide with:
L₃ containing at least one amine group (for example terminal) having reacted with the OH borne by the anomeric carbon of A,
and/or L₃ derived from a precursor having at least one halogeno group (for example bromo) having reacted with the OH groups of the Po;
   2. according to a 2nd possibility, A contains at least one residue (for example saccharide) functionalized with at least one functionalising group belonging to the group comprising the carboxylic, carboxylate, anhydride, thiol, isocyanate and epoxide functionalising groups, with:
L₃ containing at least one amine group (for example terminal) having reacted with the functionalising group(s) of A,
and/or L₃ derived from a precursor having at least one halogeno group (for example bromo) having reacted with the functionalising group(s) of A;
   3. according to a 3rd possibility, A contains at least one residue (for example POS) functionalized with at least one functionalising group belonging to the group comprising hydrogen and the groups bearing at least one ethylenic unsaturation, with L₃ having at least one group (for example terminal) bearing at least one ethylenic unsaturation having reacted with the functionalising group(s) of A;
   4. according to a 4th possibility, the first three possibilities are combined.

Advantageously, if A contains a polyol, this synthon A-X can be characterized in that said polyol is a polymer containing, for example, at least two, preferably at least 3, and still more preferably at least 10 monomer units.

The preparation of the synthon A-X can advantageously include the following essential substages:
a—reaction of the hydroxyl carried by the anomeric carbon and/or of the functionalising group or groups of A with an excess of at least one precursor of the linkage L₃ bearing a reactive end (preferably, at least one amine function—optionally terminal—and/or at least one halogeno group) capable of reacting with A;
b—elimination of the precursor.

According to a preferred characteristic, L₃ corresponds to —NH—(CH₂)_{q}≧1, with a precursor corresponding to:

NH₂CH₂_{q}C≡E

and still more preferably to propargylamine:

NH₂CH₂_{q}C≡CH

According to variants of this preferred characteristic, the precursor of the linkage L₃ could in particular be: acrylonitrile, propargyl alcohol or monopropargyl triethylene glycol.

In the case when A comprises a POS, the preparation of A-X can be carried out as described in Polymer 44 (2003) 6449-6455 Telechelic polydimethylsiloxane with terminal acetylenic groups prepared by phase transfer catalysis.

More precisely in the case of starting synthons Po-Y:

1. according to a 1st possibility, Po contains at least one saccharide with:
L₂ containing at least one amine group (for example terminal) having reacted with the anomeric carbon of the Po,
and/or L₂ derived from a precursor having at least one halogeno group (for example bromo) having reacted with the anomeric OH group or groups of the Po;
   2. according to a 2nd possibility, Po contains at least one residue (for example saccharide) functionalized with at least one functionalising group belonging to the group comprising the carboxylic, carboxylate, anhydride, thiol, isocyanate and epoxide functionalising groups with:
L₂ containing at least one amine or hydroxyl group (for example terminal) having reacted with the functionalising group(s) of the Po,
and/or L₂ derived from a precursor having at least one halogeno group (for example bromo) having reacted with the functionalising group(s) of the Po;
   3. according to a 3rd possibility, the first two possibilities are combined.

Advantageously, this synthon Po-Y can be characterized in that Po is a polymer comprising, for example, at least two, preferably at least 3, and still more preferably at least 10 monomer units.

The preparation of the synthon Po-Y can advantageously include the following essential substages:
a—reaction of the hydroxyl borne by the anomeric carbon and/or of the functionalising group or groups of Po with an excess of at least one precursor of the linkage L₂ bearing a reactive end (preferably, at least one amine function—optionally terminal—and/or at least one hydroxy function and/or at least one halo group) capable of reacting with Po;
b—elimination of the precursor;

For example, the precursor of the linkage L₂ could in particular be: H₂N(CH₂CH₂O)₃(CH₂)N₃, H₂NCH(COOH)(CH₂)₂N₃ or HO(CH₂)₆N₃. For more detail, see JACS 2005, 127, p. 14942-14949 and JACS 2004, 126, 10598-10602

More precisely in the case of starting synthons A-Y:

1. according to a 1st possibility, A contains at least one saccharide with:
L₄ containing at least one amine group (for example terminal) having reacted with the OH borne by the anomeric carbon of A,
and/or L₄ derived from a precursor having at least one halo group (for example bromo) having reacted with the OH group or groups of A;
   2. according to a 2nd possibility, A contains at least one residue (for example POS) functionalized with at least one functionalising group belonging to the group comprising the carboxylic, carboxylate, anhydride, thiol, isocyanate and epoxide functionalising groups, with:
L₄ containing at least one amine group (for example terminal) having reacted with the functionalising group or groups of A,
and/or L₄ derived from the precursor NaN₃ having reacted with the functionalising group(s) of A of the epoxide type;
and/or L₄ containing at least one halo group (for example bromo) having reacted with the functionalising group(s) of A;
   3. according to a 3rd possibility, A contains at least one residue (for example POS) functionalized with at least one functionalising group belonging to the group comprising hydrogen and the groups bearing at least one ethylenic unsaturation, with L₄ having at least one group (for example terminal) bearing at least one ethylenic unsaturation having reacted with the functionalising group(s) of A;
   4. according to a 4th possibility, the first three possibilities are combined.

Advantageously, if A comprises a polyol different from Po, this synthon A-Y can be characterized in that A is a polymer comprising, for example, at least two, preferably at least 3, and still more preferably at least 10 monomer units.

The preparation of the synthon A-Y can advantageously include the following essential substages:
a—reaction of the anomeric hydroxyl(s) and/or of the functionalising group or groups of A with an excess of at least one precursor of the linkage L₄ with or without a reactive end (preferably, at least one amine function—optionally terminal—and/or at least one halogeno group) and capable of reacting with A;
b—elimination of the precursor.

According to a preferred characteristic, A-Y is obtained from an entity A bearing functionalising groups of the epoxide type which are reacted with the precursor NaN₃.

Within the scope of this preferred characteristic, the precursor of the linkage L₄ could for example in particular be: acrylonitrile, propargyl alcohol or monopropargyl triethylene glycol.

In the case where A comprises a POS, the preparation of A-X can be carried out as described in Polymer 44 (2003) 6449-6455 Telechelic polydimethylsiloxane with terminal acetylenic groups prepared by phase transfer catalysis.

More precisely in the case of starting synthons Po-XY and A-XY, reference will be made to the descriptions of structures and preparation given above for Po-X, Po-Y, A-X and A-Y.

Stage (iii): Cycloaddition

The cycloaddition mechanism [stage (iii)] at the heart of the process according to the invention is a mechanism of 1,3-dipolar cycloaddition of a synthon Po-X or A-Y with azido reactive VII.2 units and of a synthon A-Y or Po-X with acetylenic or nitrile reactive units VII.1 (“click chemistry”) under copper-I catalysis, preferably in an aqueous, aqueous organic or organic medium.

This mechanism is particularly attractive on account of its simplicity, its non-hazardous nature for the operators and the environment, and its low cost, inter alia.

It should be noted that, according to a variant, instead of or as well as the synthons Po-X and the synthons A-Y, it is possible to use mixed synthons Po-XY each containing at least one reactive unit X and at least one reactive unit Y and mixed synthons A-XY each containing at least one reactive unit X and at least one reactive unit Y, such that these synthons Po-XY and A-XY are capable of reacting together.

Within the meaning of the invention as defined in the present document, the expression “of the order” signifies that the values concerned are given with an uncertainty of for example more or less 10%.

More precisely still, it is advisable that the cycloaddition stage (iii) be carried out in an aqueous, aqueous alcoholic or organic medium capable of solubilising and/or swelling the synthon Po-X and/or the synthon A-Y, by means of at least one metallic catalyst in ionised form, preferably Cu⁺⁺, in the presence of at least one reducing agent of Cu₊₊ to Cu⁺, in situ, this reducing agent preferably being selected from the group comprising: ascorbate, quinone, hydroquinone, vitamin K1, glutathione, cysteine, Fe²⁺, Co²⁺, applied electric potential, metal of the group comprising Cu, Al, Be, Co, Cr, Fe, Mg, Mn, Ni, and Zn, and mixtures thereof.

In practice, the metallic catalyst in ionised form, preferably Cu⁺⁺, Cu advantageously takes the form of salt(s) (ideally sulphate), still more preferably containing at least one activator comprising for example at least one salt of organic acid(s) (ideally ascorbic acid) and at least one alkali metal (ideally Na). Thus, the system CuSO4/sodium ascorbate is for example entirely suitable.

Moreover, the cycloaddition stage (iii) is preferably implemented in a reaction medium whose temperature lies between 20 and 100° C., preferably between 50 and 80° C., for 0.1 to 20 hours, preferably for 0.5 hour to 15 hours, and still more preferably for 1 to 8 hours.

The heating of the reaction medium is carried out by any appropriate means. Microwave irradiation can for example constitute an advantageous means of heating.

Advantageously, the reaction medium of the cycloaddition stage (iii) is an aqueous, aqueous organic or organic medium preferably containing at least one solvent selected from:
polar aprotic solvents, preferably dimethylformamide (DMF), dimethylacetamide (DMAc), tetrahydrofuran (THF), acetone, methyl ethyl ketone or butanone,
polar protic solvents, preferably methanol, isopropyl alcohol (IPA) or t-butanol (t-BuOH),
apolar solvents, preferably toluene, hexane, xylene,
water,
and mixtures thereof.

### Stage (iv): Separation

This being the possible separation stage (iv) of the hybrid compound Po-Ro-A from the reaction medium, it can in particular consist of carrying out:
at least one chromatography, preferably at least one chromatography on silica gel, by means of an eluent containing a mixture of a first polar solvent and at least one second less polar solvent, such as for example the mixture acetonitrile and water.
and/or at least one evaporation to dry the product.

According to another of these aspects, the present invention relates to the synthons Po-X, Po-Y, A-X, A-Y, Po-XY and A-XY according to the invention, taken as such and as defined above in the description of the process according to the invention.

The present invention also relates to the use of a hybrid compound as described above as such or as a product obtained by the process itself also defined above, as an ingredient in compositions selected from the group comprising:
detergent/surfactant compositions
shampoo compositions
soap compositions
cleaning/washing compositions, and
cosmetic compositions.

The above compositions also constitute another subject of the invention.

In particular, these compositions can be an emulsion, preferably an oil-in-water emulsion containing a hybrid compound according to the invention.

The hybrid compounds according to the invention can in particular be presented in the form of oils. They can also be presented in dispersed or solubilised form in a vector, for example at a concentration of 10 to 90% by weight. The vector can advantageously be a solvent of the polymer, for example a silicone compound, optionally volatile, for example a linear or cyclic polydimethylorganosiloxane such as cyclopentasiloxane, disiloxane, linear dimethicones, or a trimethylsiloxyphenyl dimethicone, or a mixture.

The hybrid compounds according to the invention can in particular be used as an emulsifying or co-emulsifying agent for preparing or stabilising emulsions. They can for example be used in emulsions one phase of which is a silicone oil. Presented in the form of solutions in a polyorganosiloxane, for example in cyclopentasiloxane, they can be used as an emulsifier for water-in-oil or oil-in-silicone emulsions. They can also be used to compatibilise several compounds within a formulation. They can also be used an auxiliary agent for the deposition of another compound, or as a trigger of the deposition of another compound. They can also be used as dispersant or co-dispersant agents for preparing or stabilising dispersions of particles, for example of pigments.

They can in particular be used or contained in a cosmetic formulation, whether or not intended to be rinsed, for the care of the skin and/or of the hair and/or of the lips, for example in skin care creams or milks or oils, sun protection creams or milks or oils, shampoos, conditioners, shower gels, make-up compositions, lipsticks, or deodorants. In particular, the hybrid compounds according to the invention have the advantages in these applications of being of low irritancy, partially biodegradable or bioabsorbable, of producing a pleasant feel, and/or of producing an advantageous spreading behaviour.

Other details of the invention will appear more clearly in the light of the examples given below by way of illustration.

### EXAMPLES

The hybrid compounds in the following examples are oligoorganosiloxanes or polyorganosiloxanes, more precisely PolyDiMethylSiloxanes (PDMS) with trimethylsilyl ends (MDIoM) modified with oligosaccharide groups (cf. structures A, B, C) as well as oligosaccharides modified with an alkyl chain (cf. structure D) according to a “click chemistry” mechanism.

Structure No. A: PDMS type [MD₁₀^{cellobiose modified}M]
Structure No. B: PDMS type [MD₁₀^{oligoxyloglucan modified}M]

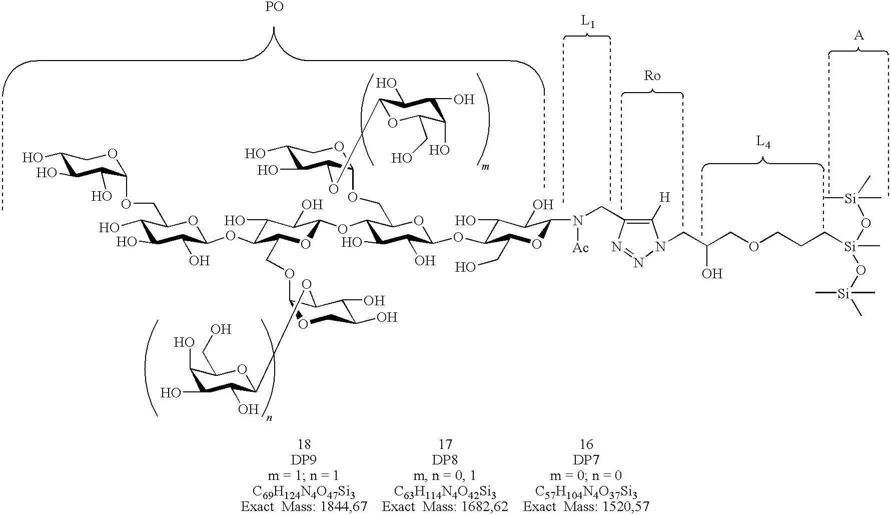

Structure No. C: PDMS type [M^{oligoxyloglucan modified}D₁₀M^{oligoxyloglucan modified}]

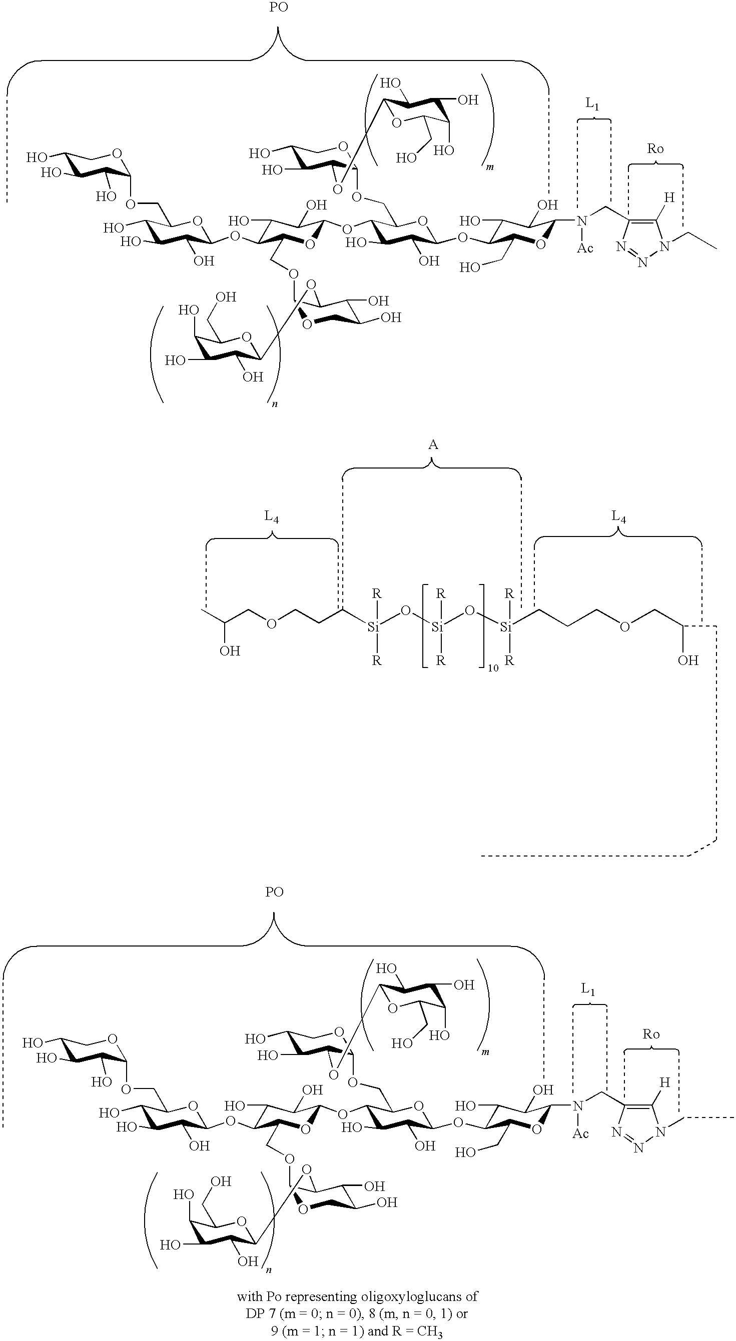

Structure No. D: Alkane (C₁₈H₃₈) modified oligoxyloglucan

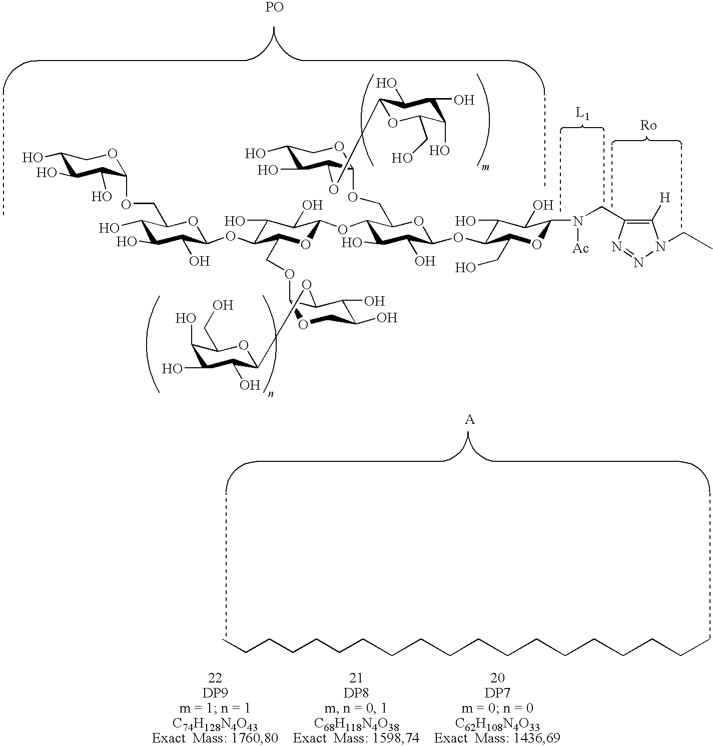

### Experimental Section

This section describes the experimental stages which made it possible to obtain the structures A, B and C described. These stages comprise:
synthesis of the terminal alkyne derivatives of the sugars,
synthesis of the azido derivatives on a polyorganosiloxane or polyalkane base,
condensation via the 1,3-dipolar cycloaddition or “click chemistry” reaction.

### Preparation of the Synthons

### Stage (I): Synthesis of the Terminal Alkyne Derivatives of the Sugars (Synthons Po-X)

### Structure A

### N-acetyl-N-propargyl-β-D-glucopyranosyl-(1→4)-β-D-glucopyranosylamine (2)

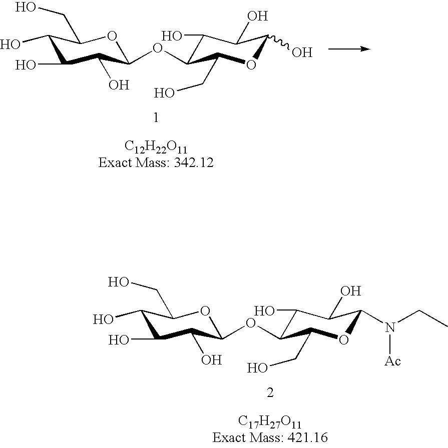

15 g of cellobiose 1 (43.8 mmol) and 62.3 ml of propargylamine (908 mmol, 21 equivs.) are placed in a 250 ml flask.

The reaction medium is continuously stirred magnetically for 40 hours at ambient temperature. Initially, the solution is heterogeneous and at the end of 16 hours becomes homogeneous. The progress of the reaction is monitored by thin layer chromatography (CH₃CN/H₂O—7:3 v/v).

The reaction medium is evaporated to dryness and co-evaporated with a mixture of MeOH and toluene (1:1 v/v) to give a yellow solid.

The solid is selectively N-acetylated by addition of 300 mL of a solution of MeOH and Ac₂O (5:1 v/v). The solution is continuously stirred magnetically for one night at ambient temperature. The solution became completely homogeneous. After evaporation to dryness and co-evaporation with the MeOH/toluene mixture (1:1 v/v), then lyophilisation, the compound 2 is obtained as a white solid (15.2 g, 36.1 mmol, 83%).

Mass spectrometry (ESI): m/z=444.07 [M+Na]⁺

¹H NMR (400 MHz, D₂O, 298K) δ (ppm)=2.28 (s, 0.8H, rotamer, CH₃(Ac)); 2.22 (s, 2.2H, rotamer, CH₃(Ac)); 3.14-4.18 (m, 14H, H-2,3,4,5,6a,6b^{GleI and GleII} and NCH₂)); 4.41 (d, 1H, J₁₋₂=7.91 Hz, H-1^{GlcII(β)}); 5.04 (d, 1H, rotamer, J₁₋₂=8.68 Hz, H-1^{GlcI(β)}); 5.50 (d, 1H, rotamer, J₁₋₂=8.86 Hz, H-1^{GlcI(β)}).

¹³C NMR (100 MHz, D₂O, 298K) δ (ppm)=18.8, 19.3 (rotamers, CH₃(Ac)); 27.8, 30.5 (rotamers, NCH₂); 57.6, 58.3 (C-6^{GlcI and GlcII}); 67.2-79.4 (C-2,3,4,5^{GlcI and GlcII}); 84.1 (C-1^{GlcII}); 100.2 (C-1^{GlcII}); 172.3, 173.5 (rotamers, C═O (Ac)).

IR (KBr): 3391 (O—H), 1645 cm⁻¹ (C═O).

### Structure B,C,D

### N-acetyl-N-propargyl-β-D-oligoxyloglucosylamine (6, 7, 8) (SYNTHONS Po-X)

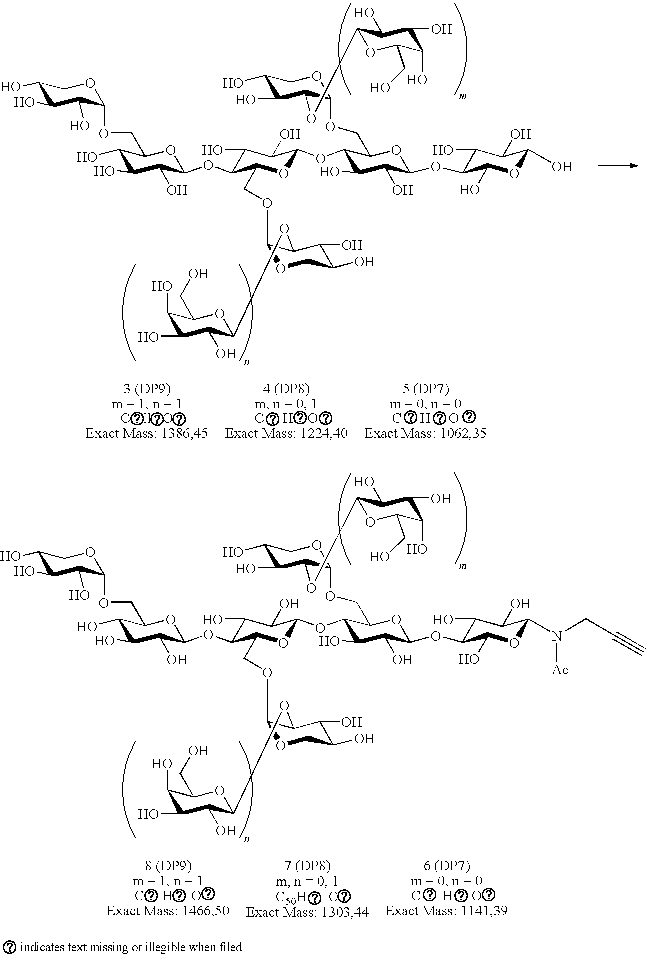

2 g of the mixture 3, 4 and 5 (1.58 mmol) (respective ratio of 0.15/0.35/10.50), 2 mL of propargylamine (31.2 mmol, 19.7 equivs.) and 3 mL of MeOH are placed in a 25 mL flask. The reaction medium is continuously stirred magnetically for 3 days at ambient temperature. The solution is quite viscous and orange in colour. The progress of the reaction is monitored by thin layer chromatography (CH₃CN/H₂O—7:3 v/v).

The solution is then diluted with 60 mL of a mixture of MeOH and CH₂Cl₂ in the proportion 1:2 v/v. A white precipitate appears spontaneously and the solution is kept stirred for 10 mins. The solution is filtered and the white solid is washed with 60 mL of the 1:2 v/v mixture of MeOH/CH₂Cl₂.

The solid is then subjected to N-acetylation by placing it in 400 mL of a solution of MeOH and Ac₂O in the proportion 20:1 v/v. The reaction medium is continuously stirred magnetically for 1 day at ambient temperature and the solution remains slightly turbid. The migration on TLC (CH₃CN/H₂O—7:3 v/v) shows no significant difference compared to the non-N-acetylated products.

The compounds 6, 7 and 8 are concentrated and lyophilised and take the form of a fluffy white powder (2 g, 1.48 mmol, 94%).

| | |
|---|---|
| Mass spectrometry (MALDI-TOF): | 6 m/z = 1163.87 [M + Na]⁺ |
| | 7 m/z = 1325.87 [M + Na]⁺ |
| | 8 m/z = 1487.84 [M + Na]⁺ |

¹H NMR (400 MHz, D₂O, 298K) δ (ppm)=2.17, 2.23 (s, CH₃(Ac)); 3.20-4.50 (m, H-2,3,4,5,6^{Glc,Gal and Xyl}); 4.60-4.90 (d and m, H-1^{Glc and Gal}); 5.18, 5.02 (d, H-1^{Xyl}); 5.44 (d, J₁₋₂=8.61, H-1^{Glc 1β}).

IR (KBr): 3402 (O—H), 1645 cm⁻¹ (C═O).

### Stage (ii): Synthesis of the Terminal Azido Derivatives

### Structure A,B

### 1,1,1,3,5,5,5-Heptamethyl-3-(1′-azido-2′-hydroxyl methoxypropyl)trisiloxane (10) (SYNTHONS A-Y)

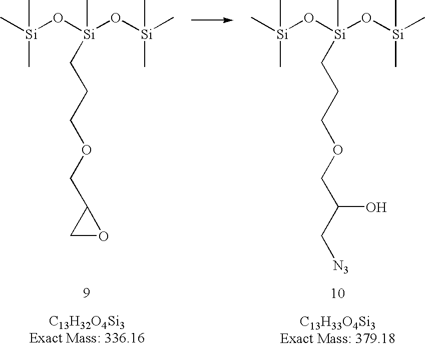

The trisiloxane 9 (12 g, 35.7 mmol) is diluted in 60 mL of isopropyl alcohol (IPA) then 5 equiv. of sodium azide (II.5 g, 178.5 mmol), 40 mL of distilled water and 20 mL of glacial acetic acid are added to attain a pH of about 6.

The reaction medium is stirred at 50° C. for 4 hrs. The reaction is monitored by TLC (9:1 v/v toluene/EtOAc).

The reaction medium is diluted with diethyl ether (200 mL) and extracted successively with a sat. solution of NaHCO₃ (2×100 mL) and water (100 mL). The organic phase is recovered, dried over Na₂SO₄, and filtered then evaporated to dryness to give the compound 10 (13.5 g, quantitative yield) in the form of a pale yellow oil which is sufficiently pure to be used for the next reaction.

Mass spectrometry (ESI): m/z=380 [M+H]⁺

¹H NMR (300 MHz, CDCl₃, 298K) δ (ppm)=−0.01 (s, 3H, SiCH₃); 0.06 (m, 18H, 2×Si(CH₃)₃); 0.42 (m, 2H, SiCH₂(α)); 1.57 (m, 2H, SiCH₂CH₂(β)); 2.57 (bs, 1H, OH); 3.40 (m, 6H, CH₂OCH₂ and CH₂N₃); 3.90 (m, 1H, CHOH).

¹³C NMR (75 MHz, CDCl₃, 298K) δ (ppm)=−0.2 (SiCH₃); 2.0 (Si(CH₃)₃); 13.7 (SiCH₂(α)); 23.4 (SiCH₂CH₂(β)); 53.8 (CH₂N₃); 69.9 (CHOH); 71.9, 74.4 (CH₂OCH₂).

IR (KBr): 3432 (O—H), 2957 and 2871 (C—H), 2102 (N₃), 1258 (C-0), 1076 and 1053 cm⁻¹ (Si-0).

### Structure C

### α,ω-Di-[1-azido-2-propanol-3-(oxypropyl)]polydimethylsiloxane (12) (SYNTHONS A-Y)

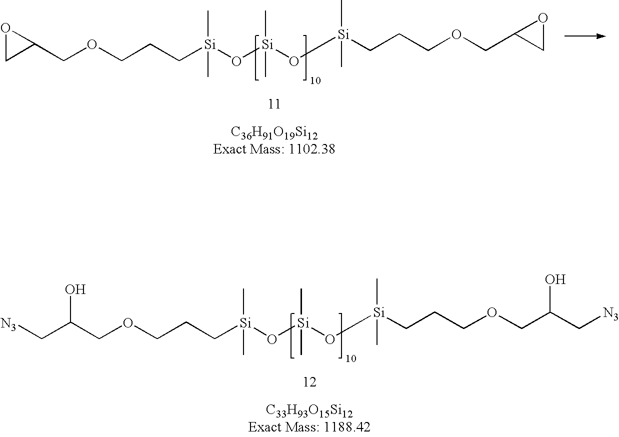

The polyorganosiloxane 11 of average DP equal to 10 (2 g, ca. 1.81 mmol) is diluted in 13 mL of isopropyl alcohol (IPA) then 5 equivs. of sodium azide (1.93 g, 9.07 mmol), 3.9 mL of distilled water and 3.3 mL of glacial acetic acid are added to reach a pH of about 6.

The reaction medium is stirred at 50° C. for 7 hrs. The reaction is followed by ¹H NMR and stopped when the starting material has been practically totally consumed.

The reaction medium is diluted with diethyl ether (30 mL) and extracted with water (10 mL). The organic phase is recovered, dried over Na₂SO₄ and filtered then evaporated to dryness to give the compound 12 (1.95 g, 89%) in the form of a colourless oil sufficiently pure to be used for the next reaction.

¹H NMR (300 MHz, CDCl₃, 298K) δ (ppm)=0.05 (m, 72H, 12×Si(CH₃)₂); 0.51 (m, 4H, 2×SiCH₂(α)); 1.58 (m, 4H, 2×SiCH₂CH₂(β)); 3.33-3.43 (m, 12H, 2×CH₂OCH₂ and CH₂N₃); 3.91 (m, 2H, 2×CHOH).

¹³C NMR (75 MHz, CDCl₃, 298K) δ (ppm)=−0.3, 1.2, 1.4 (Si(CH₃)₂); 14.3 (SiCH₂(α)); 23.5 (SiCH₂CH₂(β)); 53.7 (CH₂N₃); 69.9 (CHOH); 71.9, 74.5 (CH₂OCH₂).

IR (KBr): 3415 (O—H), 2962 and 2874 (C—H), 2104 (N₃), 1261 (C-0), 1034 and 1070 cm⁻¹ (Si—O).

### Structure D

### 1-Azido-octadecane (14) (SYNTHONS A-Y)

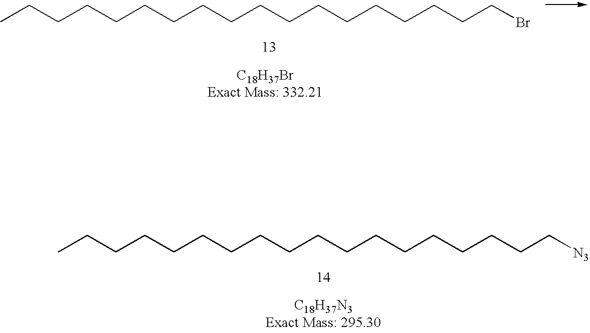

1-bromo-octadecane 13 (1 g, 3 mmol) is diluted in 10 mL of DMF and then 2 equiv. of NaN₃ (390 mg, 6 mmol) is added. The reaction mixture is heated at 50° C., with magnetic stirring for 2 hours. The reaction is monitored by TLC (eluent=petroleum ether). The DMF is removed under reduced pressure, then the residue is diluted in 20 mL of CH₂Cl₂ and extracted with 10 mL of water. The organic phase is recovered, dried over Na₂SO₄, filtered and then evaporated to dryness to give compound 14 (885 mg, quantitative yield) as a colourless liquid. This product is sufficiently pure to be used directly for the next reaction.

¹H NMR (300 MHz, CDCl₃, 298K) δ (ppm)=0.85 (t, 3H, J_{H18-H17}=6 Hz, CH₃); 1.10-1.32 (m, 30H, 15×CH₂); 1.55 (q, 2H, J_{H18-H17}=6 Hz, CH₃CH₂); 3.22 (t, 2H, J_{H1-H2}=7.5 Hz, CH₂N₃).

¹³C NMR (75 MHz, CDCl₃, 298K) δ (ppm)=14.1 (CH₃); 22.7, 26.7, 28.9, 29.2, 29.4, 29.5, 29.6, 29.7, 31.9 (CH₂); 51.5 (CH₂N₃).

IR (KBr): 2942 and 2855 (CH alkanes), 2096 cm⁻¹ (N₃).

### Stages (iii) & (iv): Condensation Compounds by “Click Chemistry”

### 4-[N-acetyl-N-(β-D-glucopyranosyl-(1→4)-β-D-glucopyranosyl)-aminomethyl]-1-[1′-(1,1,1,3,5,5,5-heptamethyl-3-(2′-hydroxyl methoxypropyl)trisiloxane)]-1H-[1,2,3]-triazole (15)

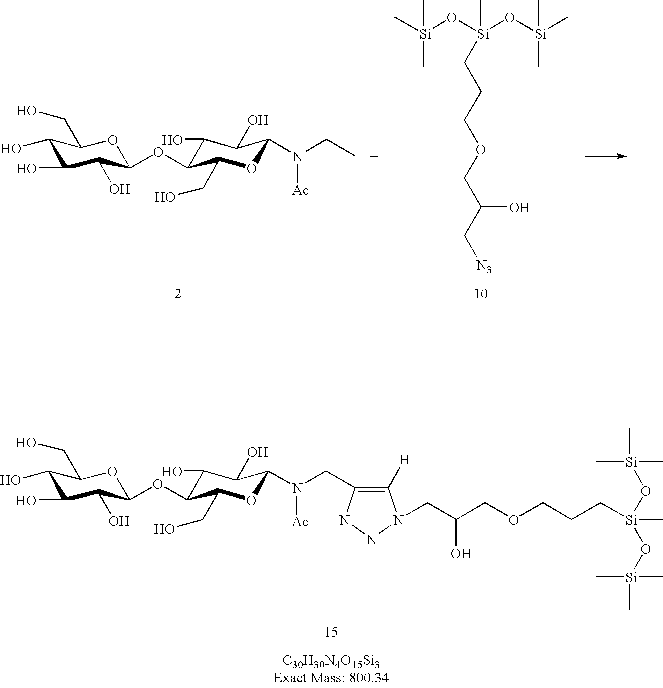

Sodium ascorbate (0.1 equivs., 4.7 mg, 24 μmol) and copper sulphate freshly dissolved in solution at 0.1M (0.01 equivs., 24 μL, 2.4 μmol) are added to a solution of the cellobiose derivative containing the terminal alkyne 2 (100 mg, 237 μmol) and the azido trisiloxane derivative 10 (1.1 equivs., 99 mg, 261 μmol) in 0.6 mL of water and 1 mL of iPrOH

The solution is brought up to 50° C. in a sealed tube and agitated for 1 hour. The reaction is monitored by TLC (CH₃CN/H₂O—7:3 v/v).

The medium is next diluted in MeOH (5 mL) then evaporated to dryness in the presence of silica.

The residue is placed on a column of silica gel. After purification by rapid chromatography on silica gel (acetonitrile/water: 9-1 v/v), the compound 15 is obtained in a yield of 89% (168 mg, 210 μmol) and in the form of a white powder after lyophilisation.

Mass spectrometry (ESI): m/z=823.46 [M+Na]⁺

¹H NMR (400 MHz, CD₃OD, 298K) δ (ppm)=0.04 (s, 3H, SiCH₃); 0.10 (m, 18H, 6×Si(CH₃)₃; 0.49 (m, 2H, SiCH₂(α)); 1.61 (m, 2H, SiCH₂CH₂(β)); 2.08, 2.22 (2×s, 3H, rotamers, CH₃(Ac)); 3.25-3.89 (m, 15H, H-2,3,4,5,6a,6b^{GlcI and GlcII} and CH₂OCH₂); 4.09 (m, 1H, CHOH); 4.40 (m, 1H, CH(OH)CH₂N); 4.44 (d, 1H, J₁₋₂=7.88 Hz, H-1^{GlcI(β)}); 4.56 (m, 1H, CH(OH)CH₂N); 4.62 (m, 2H, rotamers, CH₂N(Ac)); 5.00 (d, 0.18H, rotamer, J₁₋₂=8.21 Hz, H-1^{GlcI(β)}); 5.66 (d, 0.82H, rotamer, J₁₋₂=9.20 Hz, H-1^{GlcI(β)}); 7.86, 8.02 (s, 1H, H-5^{triazole}).

¹³C NMR (100 MHz, CD₃OD, 298K) δ (ppm)=0.0 (SiCH₃); 2.1 (Si(CH₃)₃); 14.7 (SiCH₂(α)); 22.1 (CH₃(Ac)); 24.6 (SiCH₂CH₂(β)); 37.5 (CH₂N(Ac)); 54.7 (CH(OH)CH₂^{Si}N); 61.9, 62.6 (C-6^{GlcI and GlcII}); 70.5, 71.5, 72.1, 73.3, 73.4, 75.0, 75.4, 76.6, 78.0, 78.3, 78.9, 80.0 (C-2,3,4,5^{GlcI and GlcII}, CH₂OCH₂, CHOH); 88.9 (C-1^{GlcI}); 104.7 (C-1^{GlcII}); 126.1 (C-5^{triazole}), 146.4 (C-1^{triazole}), 174.5 (C═O (NAc)).

### 4-[N-acetyl-N-(β-D-oligoxyloglucosyl)-aminomethyl]-1-[1′-(1,1,1,3,5,5,5-heptamethyl-3-(2′-hydroxyl methoxypropyl)trisiloxane)]-1H-[1,2,3]-triazole (6, 17, 18)

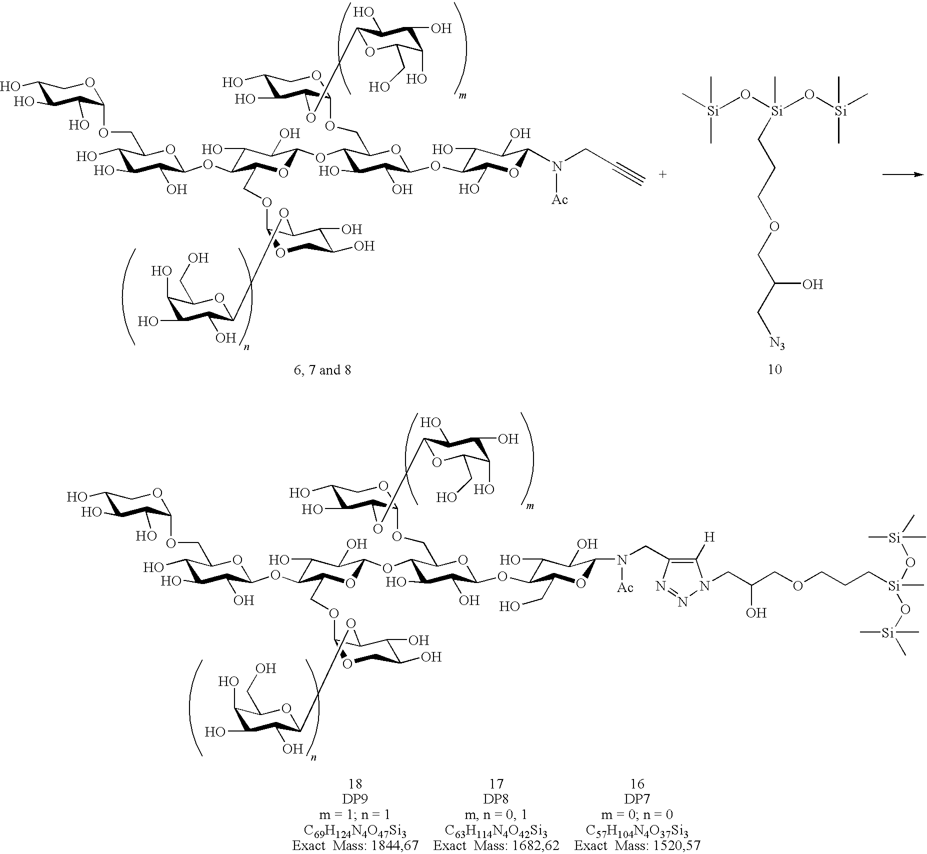

Sodium ascorbate (1 equiv., 1.6 g, 8.1 mmol) and copper sulphate freshly dissolved in 1M solution (0.5 equiv., 4.04 mL, 4.04 mmol) are added to a solution of the oligoxyloglucan derivatives containing the terminal alkyne 6(DP7), 7(DP8) and 8(DP9) (10.9 g, 8.1 mmol) and the azido trisiloxane derivative 10 (1.5 equiv., 8.4 g, 12.1 mmol) in 120 mL of water and 180 mL of iPrOH.

The solution is brought up to 50° C. in a 1 L flask and stirred for 1 hour. The reaction is monitored by TLC (CH₃CN/H₂O—7:3 v/v).

The medium is next diluted in MeOH (25 mL) then evaporated to dryness in the presence of silica. The residue is placed on a column of silica gel. After purification by rapid chromatography on silica gel (acetonitrile/water: 8-2 v/v), we observe a contamination by copper (II) of the fractions containing our compounds 16, 17 and 18, discerned by a bluish colour. These fractions are combined then concentrated and passed through a column filled with a chelating resin, Dowex M4195, previously treated with a 2M solution of NH₄OH then washed with distilled water until a pH of 7 is reached. The compounds 16, 17 and 18 are recovered by passing water through the column and are perfectly decontaminated as is shown by the colourless appearance of the solution and by measurement of the conductivity.

The compounds 16, 17 and 18 are obtained in a yield of 87% (12 g, 7 mmol) and in the form of a white powder after lyophilisation.

Mass Spectrometry (MALDI-TOF): 6 m/z=1561.51 [M+Na, H₂O]⁺ 7 m/z=1705.60 [M+Na]⁺ m/z=1723.57 [M+Na, H₂O]⁺ 8 m/z=1867.67 [M+Na]⁺

¹H NMR (400 MHz, D₂O, 298K) δ (ppm)=−0.01 (m, 21H, SiCH₃ and 6×Si(CH₃)₃); 0.35 (m, 2H, SiCH₂(α)); 1.63 (m, 2H, SiCH₂CH₂(β)); 2.12, 2.28 (2×s, 3H, rotamers, CH₃(Ac)); 3.29-3.89 (m, H-2,3,4,5,6a,6b H-2,3,4,5,6^{Glc, Gal and Xyl} and CH₂OCH₂); 4.09 (m, 1H, CHOH); 4.40-4.88 (m, CH(OH)CH₂N, CH(OH)CH₂N, CH₂N(Ac), H-1^{Glc and Gal}); 5.09, 4.97 (d, H-1^{Xyl}); 5.45 (m, H-1^{Glc 1β}); 7.86, 7.94 (s, 1H, H-5^{triazole}).

### 4-[N-acetyl-N-(β-D-oligoxyloglucosyl)-aminomethyl]-1-[α,ω-di-1-(propanol-3-(oxypropyl)polydimethylsiloxane)]-1H-[1,2,3]-triazole (12)

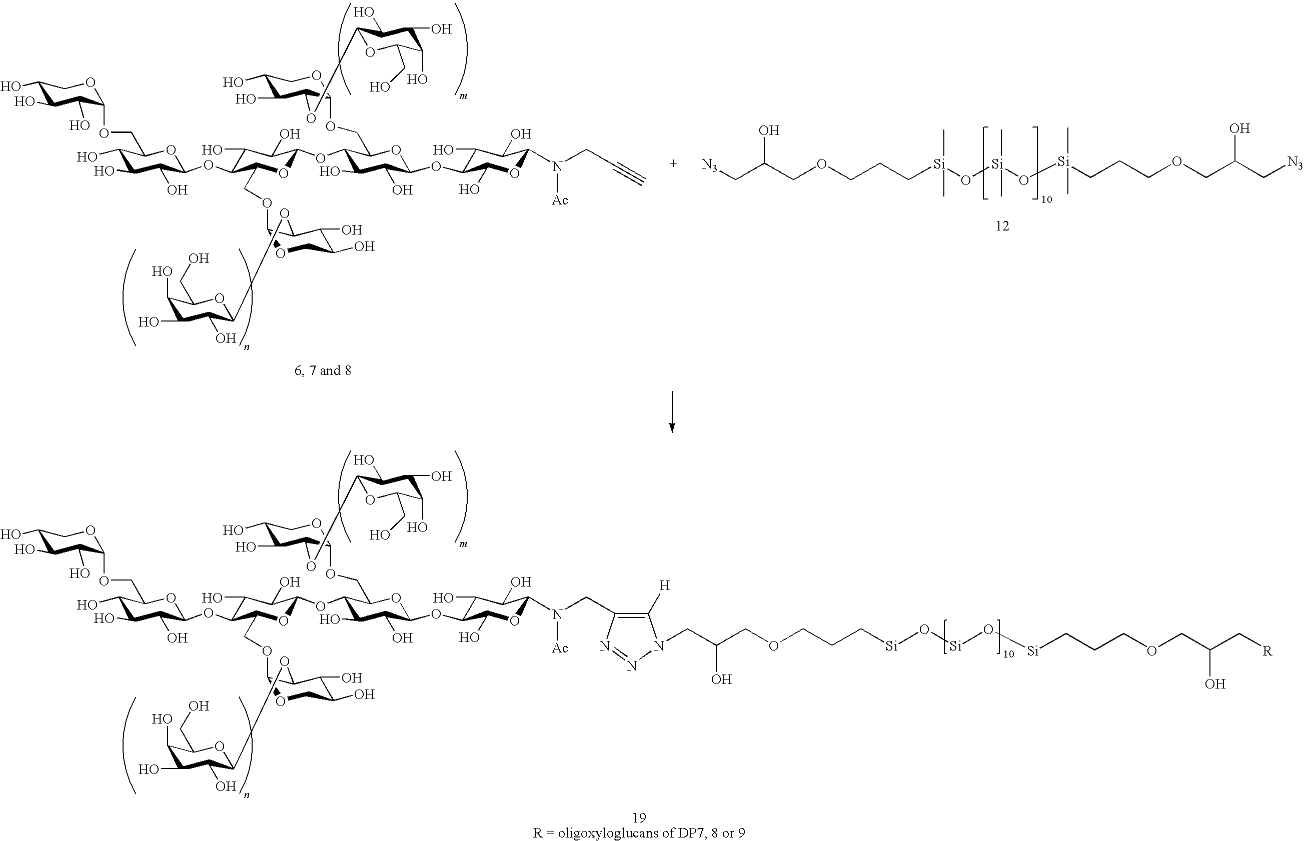

Sodium ascorbate (2 equivs., 73.4 mg, 370 μmol) and copper sulphate freshly dissolved in 1M solution (1 equiv., 185 μL, 185 μmol) are added to a solution of the oligoxyloglucan derivatives containing the terminal alkyne 6(DP7), 7(DP8) and 8(DP9) (2 equivs., 500 mg, 370 μmol) and the polyorganosiloxane derivative 12 of average DP 10 (220 mg, 185 μmol) in 3 mL of water and 5 mL of iPrOH.

The solution is brought up to 50° C. in a sealed tube and shaken for 1 hour. The reaction is monitored by TLC (CH₃CN/H₂O—7:3 v/v).

The medium is next diluted in MeOH (25 mL) then evaporated to dryness in the presence of silica. The residue is placed on a column of silica gel. After purification by rapid chromatography on silica gel (acetonitrile/water: 8-2 v/v), the compounds 19, consisting of a multitude of combinations of condensation products and impossible to determine, are obtained with a mass yield of 81% (583 mg) and in the form of a white powder after lyophilisation.

¹H NMR (300 MHz, D₂O, 298K) δ (ppm)=−0.07 (m, 57H, ˜9.5×Si(CH₃)₂); 0.35 (m, 4H, 2×SiCH₂(α)); 1.49 (m, 4H, 2×SiCH₂CH₂(β)); 2.03, 2.18 (2×s, rotamers, CH₃(Ac)); 3.05-4.88 (m, H-2,3,4,5,6^{Glc, Gal and Xyl} and CH₂OCH₂^{Si}, CHOH^{Si}, CH(OH)CH₂N, CH(OH)CH₂N, CH₂N(Ac), H-1^{Glc and Gal}); 4.97-5.09 (m, H-1^{Xyl}); 5.32 (m, H-1^{Glc 1β}); 7.82, 7.93 (s, 1H, H-5^{triazole}).

IR (KBr): 3383 (OH), 2961 (C—H), 1644 (C═O), 1261 (C—O), 1044 and 1090 cm⁻¹ (Si—O).

### 4-[N-acetyl-N-(β-D-oligoxyloglucosyl)-aminomethyl]-1-(1-octadecane)-1H-[1,2,3]-triazole (20, 21 and 22)

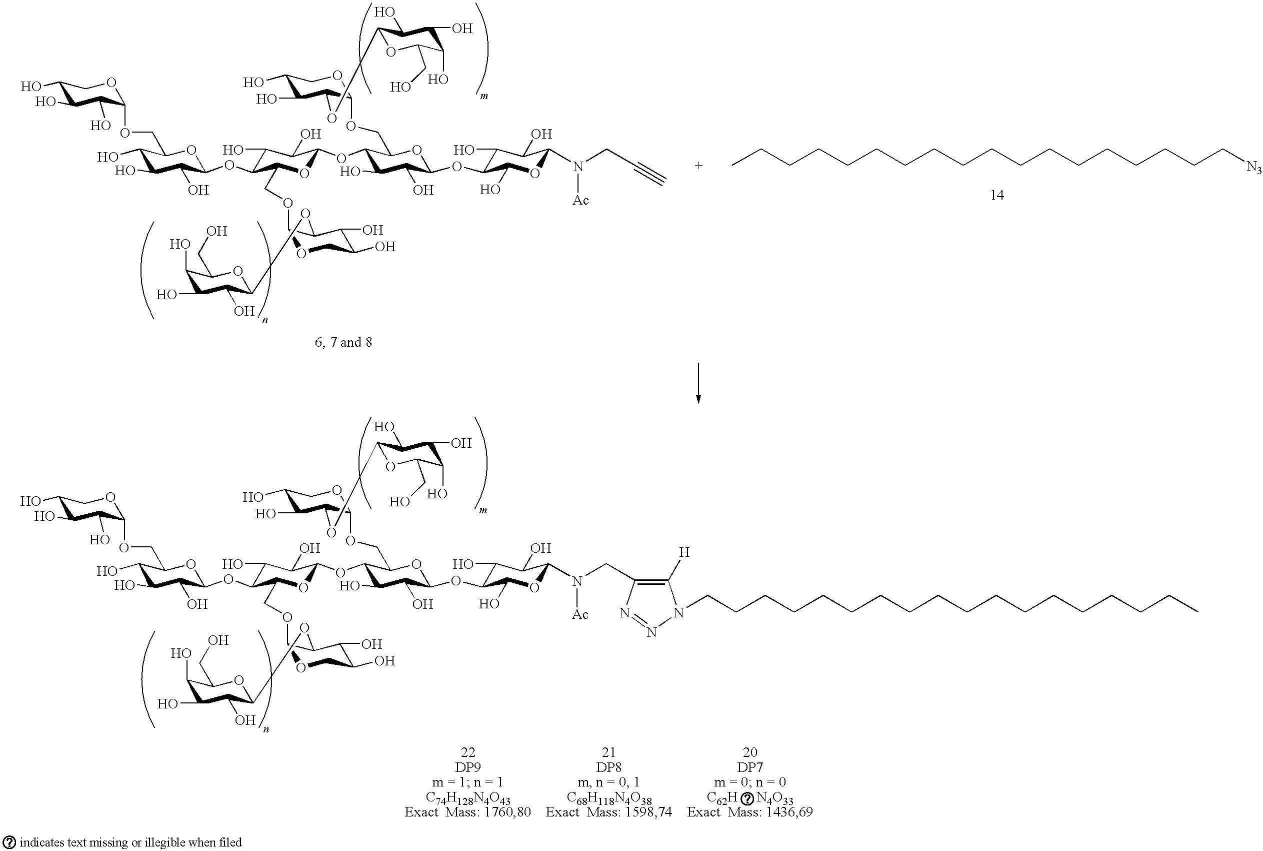

Sodium ascorbate (1 equiv., 29 mg, 150 μmol) and copper sulphate freshly dissolved in 1M solution (0.5 equiv., 75 μL, 75 μmol) are added to a solution of the oligoxyloglucan derivatives containing the terminal alkyne 6(DP7), 7(DP8), 8(DP9) (200 mg, 150 μmol) and 1-azido-octadecane 14 (1.1 equiv., 48 mg, 160 μmol) in 1.5 mL of water and 3 mL of iPrOH.

The solution is brought up to 50° C. in a sealed tube and agitated for 1 hour. The reaction is monitored by TLC (CH₃CN/H₂O—7:3 v/v).

The reaction mixture is next diluted in MeOH (10 mL) then evaporated to dryness in the presence of silica. The residue is placed on a column of silica gel. After purification by rapid chromatography on silica gel (acetonitrile/water: 8-2 v/v), the compounds 20, 21 and 22 are obtained with a yield of 81% (180 mg, 121 μmol) and in the form of a white powder after lyophilization.

¹H NMR (400 MHz, D₂O, 298K) δ (ppm)=0.76 (m, 3H, CH₃); 1.10-1.32 (m, 30H, 15×CH₂); 1.63 (m, 2H, CH₃CH₂); 1.90, 2.08 (2×s, 3H, rotomers, CH₃(Ac.)); 3.12-4.86 (m, H-2,3,4,5,6^{Glc, Gal and Xyl}); 4.75 (m, H-1^{Glc and Gal}); 5.08 (d, H-1^{Xyl}); 7.77, 7.90 (s, 1H, H-5^{triazole}).

IR (KBr): 3402 (OH), 2921, 2851 (C—H).

1. Hybrid compound Po-Ro-A containing at least one polyol entity (Po) and in which at least one of the atoms of Po is substituted with at least one group of the following general formula (I):
-Ro-A (I)
in which:
Ro is a linking hinge of the following formula (II.1) or (II.2): 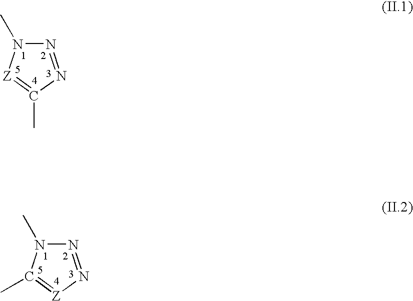
with Z representing a carbon or a nitrogen atom;
the polyol entity Po is selected from the polymeric synthetic non-saccharide polyols, or from the saccharides (hydrogenated or not) containing at least two, preferably at least three monosaccharide units,
A is an inorganic or organic entity, optionally polymeric; and if there is a plurality of entities A per molecule of hybrid compound, said entities A are mutually identical to or different, the organic entity A being selected or derived from a compound selected from the group comprising:
synthetic polymers, their copolymers or monomer units making it possible to obtain them
alkyls, alkenyls, alkynyls, aryls and combinations of the latter
and combinations thereof.

2. Compound according to claim 1, characterized in that at least one of the hydroxyl functions of the polyol entity (Po) is substituted with at least one grouping with the following general formula (I): -Ro-A as defined in claim 1.

3. Hybrid compound according to claim 1, characterized in that the hinge Ro or at least one of the hinges Ro is linked to the entity Po and/or to the entity A by a divalent linkage -L-, L preferably being a hydrocarbon unit or an atom such as O or S.

4. Hybrid compound according to claim 1, characterized in that none of the hinges Ro is linked to the entity Po by a divalent linkage -L- and in that said hybrid compound has at least one entity A free from amino acid(s) and/or peptide(s) and/or their analogue(s) and/or derivative(s).

5. Hybrid compound according to claim 1, characterized in that the entity A is selected or derived from a compound selected from the group comprising:
synthetic polymeric non-saccharide polyols, their copolymers or the monomer units making it possible to obtain them;
polyorganosiloxanes (POS), their copolymers or the monomer units making it possible to obtain them;
polyalkylene glycols (or else alkylene polyoxides), preferably polyethylene glycols (or else ethylene polyoxides) and/or polypropylene glycols (or else propylene polyoxides), and/or polytetraethylene glycols, and/or copolymers or co-oligomers, in particular random or block copolymers or co-oligomers, thereof or of polypropylene glycols and of polypropylene glycols (or else random or block ethylene and propylene polyoxides), these polyalkylene glycols optionally being functionalised with or onto other groups, for example with or onto amine groups (Jeffamines), and/or optionally being terminated at least one end by a hydroxyl group or by an alkyl group, for example a C₁-C₃₀ alkyl;
polyamides, copolymers thereof or monomer units making it possible to obtain them;
polyesters, copolymers thereof or monomer units making it possible to obtain them;
polybutadienes, copolymers thereof or monomer units making it possible to obtain them;
polystyrenes, copolymers thereof or monomer units making it possible to obtain them;
optionally, alkyls, alkenyls, alkynyls, aryls and combinations of these;
inorganic substances other than silica;
and combinations thereof.

6. Hybrid compound according to claim 1, characterized in that the free valence bond of the nitrogen at the position 1 in formulae (II.1) and (II.2) links the hinge Ro to Po and the free valence bond of the carbon or of the atom Z at the 4 or 5 position in formulae (II.1) and (II.2) links the hinge Ro to A.

7. Hybrid compound according to claim 1, characterized in that the free valence bond of the nitrogen at the 1 position in formulae (II.1) and (II.2) links the hinge Ro to A and the free valence bond of the carbon or of the atom Z at the 4 or 5 position in formulae (II.1) and (II.2) links the hinge Ro to Po.

8. Hybrid compound according to claim 1, characterized in that the polyol entity Po or the polyols that can be contained in the entity A is (are) selected:
from the synthetic, polymeric, non-saccharide polyols,
and/or from the saccharides (hydrogenated or not) containing at least two, preferably at least three monosaccharide units.

9. Hybrid compound according to claim 1, characterized in that A contains at least one POS bearing siloxy units M, D, T and/or Q, preferably at least one POS bearing siloxy units M and D, optionally T and/or Q, and still more preferably at least one POS of type M(D)_{d}M, M(D)_{d}(T)ₜM, MQ, with d,t being rational numbers greater than or equal to 0.

10. Hybrid compound according to claim 1, characterized in that it corresponds to at least one of the following formulae 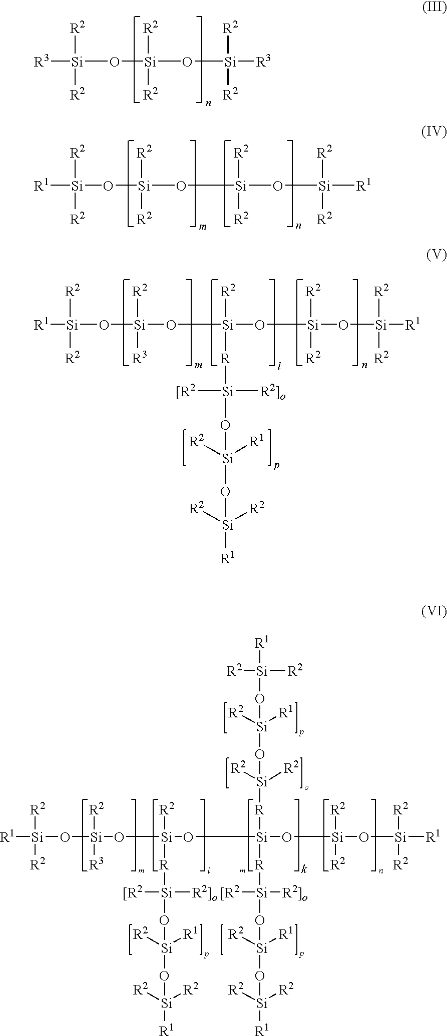
in which:
R², identical or different, is a hydrocarbon group, preferably a methyl group,
R³, identical or different, is a group of formula -Ro-Po in which Ro and Po are as defined in any one of claims 1 to 7,
R¹, identical or different, is a group R² or R³,
R is a divalent group comprising an oxygen atom, preferably an —O— group,
m is an average number different from 0,
n is an average number greater than or equal to 0,
k and l are average numbers greater than or equal to 0, and
o and p, identical or different, are average numbers greater than or equal to 0.

11. Process for the preparation in particular of the hybrid compound according to claim 1, characterized in that
i. a synthon Po-X, containing at least one reactive unit X having at least one reactive end of formula (VII.1): —C≡E; with E=CH or N is used and/or is prepared;
ii. a synthon A-Y containing at least one reactive unit Y having at least one reactive end of formula (VII.2); —N₃; the reactive end (VII.2) being capable of reacting with the reactive end (VII.1) is used and/or is prepared;
iii. the synthon Po-X is reacted with the synthon A-Y according to a cycloaddition mechanism, so as to obtain a hybrid compound Po-Ro-A containing at least one polyol entity (Po) in which at least one of the hydroxyl functions of Po is substituted with at least one grouping of the following general formula (I′): -Ro-A; with Ro and A as defined in any one of claims 1 to 10;
iv. optionally, Po-Ro-A is separated from the reaction mixture so that it can be recovered.

12. Process according to claim 11, characterized in that the cycloaddition stage (iii) is carried out in an aqueous, aqueous alcoholic or organic medium capable of solubilising and/or swelling the synthon Po-X and/or the synthon A-Y, by means of at least one metallic catalyst in ionised form, preferably Cu⁺⁺, in the presence of at least one reducing agent of Cu⁺⁺ to Cu⁺, in situ, this reducing agent preferably being selected from the group comprising: ascorbate, quinone, hydroquinone, vitamin K1, glutathione, cysteine, Fe²⁺, Co²⁺, applied electrical potential, metal of the group comprising Cu, Al, Be, Co, Cr, Fe, Mg, Mn, Ni, and Zn, and mixtures thereof.

13. Process according to claim 11, characterized in that the reaction medium contains at least one solvent selected from:
polar aprotic solvents, preferably dimethylformamide (DMF), dimethylacetamide (DMAc), acetone, methyl ethyl ketone or butanone
polar protic solvents, preferably methanol, isopropyl alcohol (IPA) or t-butanol (t-BuOH),
apolar solvents, preferably toluene, hexane or xylene,
water,
and mixtures thereof.

14. Process according to claim 11, characterized in that the optional separation (iv) of Po-Ro-A from the reaction mixture in particular consists of carrying out:
at least one chromatography, preferably at least one chromatography on silica gel, by means of an eluent containing a mixture of a first polar solvent and of at least one second less polar solvent, such as for example the mixture acetonitrile and water
and/or at least one evaporation to dry the product.

15. Synthon Po-X characterized in that it comprises at least one reactive unit X having at least one reactive end of formula (VII.1.1):
-[L₁]ₐ-C≡E
with E=CH or N, a=1, said end being linked to the residue Po by a L₁ which is a divalent hydrocarbon linkage.

16. Synthon Po-X according to claim 15, characterized in that:
1. according to a 1st possibility, Po contains at least one saccharide with:
L₁ containing at least one amine group (for example terminal) having reacted with the anomeric carbon of the Po,
and/or L₁ derived from a precursor having at least one halogeno group (for example bromo) having reacted with the OH group or groups of the Po;
2. according to a 2nd possibility, Po contains at least one residue (for example saccharide) functionalized with at least one functionalising group belonging to the group comprising the carboxylic, carboxylate, anhydride, thiol, isocyanate and epoxide functionalising groups with:
L₁ containing at least one amine group (for example terminal) having reacted with the functionalising group(s) of the Po,
and/or L₁ derived from a precursor having at least one halogeno group (for example bromo) having reacted with the functionalising group(s) of the Po;
3. according to a 3rd possibility, the first two possibilities are combined.

17. Synthon Po-X according to claim 15, characterized in that Po is a polymer.

18. Mixed synthon Po-XY with Po as defined in claim 1, characterized in that it has at least one reactive unit X as defined in claim 15 and at least one reactive unit Y having at least one reactive end of formula (VII.2.1):
-[L₂]ₐ-N₃
with a=1; said end being linked to the residue Po by a linkage L₂ which is a divalent hydrocarbon linkage.

19. Use of a hybrid compound according to claim 1, as ingredients in compositions selected from the group comprising:
detergent/surfactant compositions,
shampoo compositions,
soap compositions,
cleaning/washing compositions,
cosmetic compositions.

20. Compositions according to the use in claim 19.

21. Emulsion, preferably oil-in-water emulsion, containing a hybrid compound according to claim 1.

22. Use of a hybrid compound according to claim 11, as ingredients in compositions selected from the group comprising:
detergent/surfactant compositions,
shampoo compositions,
soap compositions,
cleaning/washing compositions,
cosmetic compositions.

23. Compositions according to the use in claim 22.

24. Emulsion, preferably oil-in-water emulsion, containing a hybrid compound obtained by the process according to claim 11.
